(19) United States
(12) Patent Application Publication
(10) Pub. No.: US 2012/0029417 A1
(43) Pub. Date: Feb. 2, 2012

(54) COSMETIC TREATMENT METHOD INCLUDING THE PROJECTION OF AN IMAGE ONTO THE ZONE TO BE TREATED

(75) Inventors: Henri Samain, Bievres (FR); Franck Giron, Ferrieres en Brie (FR)

(73) Assignee: L'OREAL, Paris (FR)

(21) Appl. No.: 13/147,017

(22) PCT Filed: Feb. 23, 2010
(86) PCT No.: PCT/IB2010/050782

§ 371 Date: Oct. 7, 2011

Related U.S. Application Data
(60) Provisional application No. 61/158,764, filed on Mar. 10, 2009.

(30) Foreign Application Priority Data
Feb. 23, 2009 (FR) 09 00822

(51) Int. Cl. A61M 35/00

(52) U.S. Cl. 604/20

(57) ABSTRACT

A treatment method for treating human keratinous material, the method including: a) projecting an image onto a zone to be treated; b) modifying the content and/or adjusting the projected image; and c) cosmetically treating the zone as a function of the image as modified and/or adjusted in this way.

The present invention relates to the treatment of human keratinous material in particular making up the skin and other human keratinous material, e.g. the lips, the nails, or the hair.

### PRIOR ART

When making up, it is usual to use a colored substance that is deposited on the body or face.

The final result not only depends on the quality of the products used, in particular the ingredients and the formulation techniques employed, but also on the dexterity of the user.

When a person applies makeup, attempts are made to position the various strokes of color correctly.

If this operation is performed poorly, the result may lose much of its esthetic aspect. It thus becomes necessary to correct the makeup, or even to remove it in order to recommence the operation.

In addition to the fact that it is not easy to apply makeup with precision, some particular situations may render the operation even more problematic, e.g. a lack of general experience in the art of making up, the desire to change makeup style, muscular stiffness or disabilities in the arms or the hands, problems with vision or with trembling, or problems of three-dimensional visualization.

For some people, the problem of making up is exacerbated by the fact that the makeup zones may be irregular, bumpy, or poorly defined, which is typical for the lips and the eyelids of elderly people. It is thus difficult for such people to create a makeup look that presents curves that are uniform and harmonious.

In addition, some people desire makeup effects that are fairly sophisticated, requiring hand movements that are precise and complex, and any help in performing the makeup operations would be useful.

Another problem exists for people who wish to makeup thin lips by going beyond the zone of the lips in order to render fuller lips. It is difficult to achieve such extended makeup since the person does not have a reference point on which to rely.

The problem of making up also exists for professional makeup artists. Often, it is not easy to perform uniform hand movements because of the movements of the person being made-up.

The impact of poorly-positioned makeup depends in part on the technique used. A composition that uses a wide applicator, e.g. a blusher brush, poses few problems since the applicator produces a stroke that is quite fuzzy. The esthetic impact of a positioning error is thus limited. The opposite applies with compositions that use a fine applicator, e.g. like those for drawing a line, for which the esthetic impact of a positioning error is high.

There thus exists a need to benefit from means for applying makeup in fine and precise manner while limiting the risks of poor positioning.

In addition, positioning is made more or less difficult depending on the low or high visibility of the color imparted. If the color can be seen clearly, positioning is easier, but if the color is little saturated, positioning is more difficult since the user may have difficulty in seeing what has been done.

It has been discovered that it is possible to obtain satisfactory makeup results using light-sensitive makeup. The precision of the result exceeds that which users normally obtain with conventional makeup, without the need either for particular dexterity or for training.

Further, light-sensitive makeup may produce color effects that go beyond what is normally accepted for makeup. This may be any pattern imitating a conventional makeup pattern, or a text, logo, etc.

Light-sensitive makeup is based on using at least one thermally stable photochromic composition that is capable of being developed by light radiation, for example UV radiation, and that retains a change in appearance linked to irradiation for at least one hour.

In order to create a light-sensitive makeup look, at least one thermally stable photochromic composition is deposited on the zone to be treated in the form of at least one layer.

When the thermally stable photochromic composition is applied, it is in the non-developed state and it may be colored or colorless, depending on the ingredients used.

Irradiation of the layer of thermally stable photochromic composition may be carried out selectively, by irradiating in a non-uniform manner. Thus, certain regions need not be developed while others are, and/or some regions may be developed to varying extents, leading to different intensities of color.

The light energy used remains relatively low and does not cause the skin to tan.

One method of making up with light-sensitive makeup is described in patent EP-A-0 938 887, which is incorporated by reference and which employs thermally stable photochromic agents that are applied to the skin. That patent describes a photochromic agent selected from diarylethenes and fulgides.

The method of making up with light-sensitive making up poses positioning problems since, depending on the location that is illuminated, the makeup that is going to appear risks being poorly positioned.

The problem also exists with cosmetic treatments that use other colorant systems, such as those developing a color by chemical, biological, enzymatic, or physical reaction, e.g. DHA, oxidizing colorants, or polyphenols that develop in oxygen from the air.

The problem may also exist with treatments other than treatments seeking to impart a color. For example some care products require a certain amount of precision in positioning, e.g. decoloring, depigmenting, or depilatory compositions.

United States application US-2007/0038270-A1 discloses various methods in which a photosensitive composition is deposited on the skin in a pattern or is exposed to light corresponding to a pattern to be produced.

Application WO 2008/144787 discloses a method of making visible patterns on human skin. In that method, an addressable matrix imager emitting UV light is used to tan the skin selectively.

### SUMMARY OF THE INVENTION

Exemplary embodiments of the invention provide a treatment method for treating human keratinous material, the method comprising the steps consisting in:
projecting an image onto a zone to be treated;
possibly modifying the content and/or adjusting the projected image; and
treating, in particular cosmetically treating, the zone as a function of the projected image.

Modification of the content or adjustment may take place manually or automatically.

Adjustment may comprise focusing the image and/or positioning the image on the face.

Modification of the content may comprise modifying the pixels of the image when said image is a pixellated image. Modification may comprise a change in the light spectrum that is used to form the image.

By means of the invention, it is possible to obtain precise positioning for a light-sensitive makeup and/or a makeup or for a care product, without particular effort on the part of the person who applies the makeup, the care product, or the light-sensitive makeup.

The projected image may be used to position subsequent treatment which may advantageously, but not exclusively, be a light-sensitive makeup.

The projected image may also be used to define the zone to be treated.

In this event, the projected image may advantageously include one or more reference points, and the treatment may be performed as a function of the reference point(s).

The method may include, before step a), applying a thermally stable photochromic composition on the zone to be treated with a view to creating a light-sensitive makeup for the purpose of making up during subsequent treatment using the light-sensitive makeup, or for the purpose of drawing reference points that are useful for subsequent treatment other than making up with a light-sensitive makeup.

In exemplary implementations of the invention, the light forming the projected image may initially be selected so as not to activate the thermally stable photochromic composition, and the method may thus comprise the steps consisting in:

i) optionally the content and/or adjusting the image projected on the zone to be treated; and

ii) irradiating the zone so as to excite the thermally stable photochromic composition as a function of the image projected in step i).

The light may be modified between steps i) and ii), and a source of UV and/or near UV light may be used in step ii).

The thermally stable photochromic composition may be irradiated in such a manner as to create at least one reference point and/or one outline, in particular a template for applying makeup or a care product. The zone may thus be treated using the reference point or the outline.

Treatment may comprise manually applying a makeup as a function of the reference point(s) and/or the outline(s). Treatment may also be carried out by automatically detecting the reference point(s) and/or the outline(s), in particular by using an appliance that is positioned and/or moved over the zone to be treated.

In exemplary implementations of the invention, any movement of the zone to be treated is detected and the projected image is modified automatically as a function of the movement thus detected of the zone to be treated during projection of the image. This makes it possible to reduce the risk of blurring the resulting light-sensitive makeup look.

The projected image is advantageously produced by an electronic addressable matrix imager.

The projected image may be generated from at least one acquired image of the person to receive the treatment. This makes it possible to create a light-sensitive makeup that is adapted to the morphology of the person being treated.

The projected image may take account of distortions induced by the relief of said zone. For this purpose, it is possible to perform a prior acquisition of the relief of the zone that is to be treated.

An animated sequence of images may be projected that are suitable for guiding the user in applying makeup.

The projected image may be an image simulating the application of makeup, or at least one reference point and/or one outline. The outline may be created by a continuous or a discontinuous line, or by the edge of a surface contrasting with its neighborhood.

### Thermally Stable Photochromic Composition

In accordance with the invention, the light-sensitive makeup look is created using a suitable thermally stable photochromic composition.

The thermally stable photochromic composition of the invention contains one or more thermally stable photochromic agents that are suitable for creating a light-sensitive makeup look, i.e. they change appearance under the influence of light radiation.

The thermally stable photochromic agent or agents used in the invention may be thermally stable photochromic agents or irreversible photochromic agents, i.e. once the change in appearance is obtained, it is permanent.

Depending on the thermally stable photochromic agent or agents used, the light-sensitive makeup look may be created by progressively developing said thermally stable photochromic agent or agents by exposure to suitable radiation, for example UV and/or near UV, or by starting from a thermally stable photochromic composition comprising one or more thermally stable photochromic agents in the already developed state that are brought into a non-developed state by the application of suitable radiation, for example visible near-UV visible.

The light-sensitive makeup may implement both development of one or more thermally stable photochromic agents and erasure of one or more thermally stable photochromic agents, for example successively or alternately, in order to obtain precisely the desired makeup result.

The thermally stable photochromic composition may be contained, with the thermally stable photochromic agent or agents in the non-developed state, in a packaging device before application to the keratinous material. In this configuration, the thermally stable photochromic composition may be applied to the keratinous material with the thermally stable photochromic agent or agents in the non-developed state, then the radiation may change said photochromic agent or agents into the developed state is applied.

In a variation, the photochromic composition is applied to the keratinous material with the thermally stable photochromic agent or agents in the non-developed state, then they are brought into a developed state, and thereafter radiation is applied selectively to change the thermally stable photochromic agent or agents into the non-developed state, for example in a localized manner, in order to create one or more patterns, for example, and/or to obtain the desired color.

In another variation, the thermally stable photochromic composition is contained in a packaging device with the thermally stable photochromic agents being in the developed state. The thermally stable photochromic composition is then applied with the thermally stable photochromic agent or agents being in the developed state, and these are brought into a non-developed state in a selective manner in order to form one or more patterns and/or to obtain the desired color.

When seeking to use a thermally stable photochromic composition in which the thermally stable photochromic agent or agents is/are already in the developed state when the composition is applied to the keratinous material, it is optionally possible to use a packaging device including a light source suitable for exposing the thermally stable photochromic composition to light radiation, for example within the enclosure of the receptacle containing it or at a distribution orifice or an application member, which light radiation is of a wavelength suitable for developing the thermally stable photochromic agent or agents.

The thermally stable photochromic composition may comprise a thermally stable photochromic agent capable, for example, of generating a color in the developed state, and, for example, that is colorless in the non-developed state, or a mixture of thermally stable photochromic agents producing respectively different colors in the developed state while having another color or being colorless in the non-developed state.

As an example, it may be possible to use a thermally stable photochromic composition comprising a mixture of respective yellow, blue, and magenta thermally stable photochromic agents with, for example, a larger proportion of thermally stable photochromic agent of color that is yellow in the developed state, the proportions being selected, for example when all of the thermally stable photochromic agents are in the developed state, to obtain a hue close to that of the skin. Thus, in one example of the invention, a mixture of respectively yellow, magenta, and blue thermally stable photochromic agents is used in relative proportions of approximately 50%, 35% and 15%.

When the thermally stable photochromic composition comprises a plurality of thermally stable photochromic agents, it is possible to use thermally stable photochromic agents capable of being developed by exposure to radiation with respective different dominant wavelengths so that, by selecting the wavelength of the radiation to which the thermally stable photochromic composition is exposed, it is possible to develop one color rather than another. It is also possible to use thermally stable photochromic agents in a thermally stable photochromic composition capable of being erased when exposed to the respective dominant wavelengths, which means that, by selecting the characteristics of the light used to erase the thermally stable photochromic composition, a given color may be erased rather than another.

### Measurement of the Thermal Stability of a Thermally Stable Photochromic Agent

The test to determine whether a photochromic agent is thermally stable is as follows. The agent to be tested, initially with color Eᵢ in the non-developed state, is irradiated using UV radiation for 1 minute at 1 J/cm² [joule per square centimeter], then its final color E_{f} is determined using a spectrocolorimeter, for example that from MINOLTA CM 2002 (d/8, SCI, D65, 2° observer); a color difference

ΔE_{i,f}={square root over ((a_{f}−aᵢ)²+(b_{f}−bᵢ)²+L_{f}−Lᵢ)²)}

is obtained in CIE Lab space, which corresponds to the maximum development. Said compound is then left in total darkness for 60 minutes at 25° C., then its color Eᵣ is determined using the above method. If the new value of ΔE_{i,r} is at least 50% of the value of ΔE_{i,f} corresponding to the maximum development, it is considered that the compound is thermally stable. Preferably, the thermally stable photochromic agent is selected so that once developed, the makeup obtained may be visually conserved for more than one hour, preferably more than four hours.

The thermally stable photochromic composition may be free of thermally reversible photochromic compounds such as doped titanium oxide, spiropyrans, spirooxazines, or chromenes, unless certain forms of those compounds fall within the definition of thermally stable photochromic agents.

The thermally stable photochromic agent or agents of the invention are advantageously such that under an initial irradiation I₁, this or these agents are developed by becoming colored, starting from a substantially colorless or faintly colored state; and under a second irradiation I₂ that differs from the first, this or these agents go back to being substantially colorless or faintly colored. In implementations of the invention, the irradiation I₁ is UV irradiation (290 nm to 400 nm), in particular UVA (320 nm to 400 nm) and/or UVB, preferably in the near UV (400 nm to 440 nm), while irradiation I₂ is irradiation in the visible, for example white light.

### Thermally Stable Photochromic Agents

Preferred examples of photochromic agents that may be used are compounds which belong to the diarylethene family and those which belong to the fulgide family; this list is not limiting, however. The skilled person may make reference to patent EP-A-0 938 887 that describes examples of thermally stable photochromic agents.

Diarylethenes may be represented by the following formula (I):

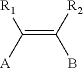

in which the radicals R₁ and R₂ are always “cis” relative to the double bond.

These radicals R₁ and R₂, independently of each other, may be selected from C₁-C₁₆ alkyl radicals, which may be fluorinated or perfluorinated, and nitriles.

Compounds with the following formula may be mentioned in particular:

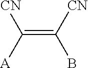

They may also form a cycle containing 5 or 6 carbon atoms, which may be fluorinated or perfluorinated, in particular with the following formula:

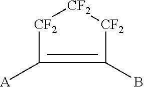

or
form a 5 carbon atom anhydride cycle, in particular with the following formula:

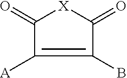

in which X may be an oxygen atom or a —NR₃ radical, where R represents a C₂-C₁₆ alkyl and/or hydroxyalkyl radical.

Radicals A and B may also be equal or different and in particular may represent a 5-atom cycle or a 5- or 6-atom bi-cycle with the following structures:

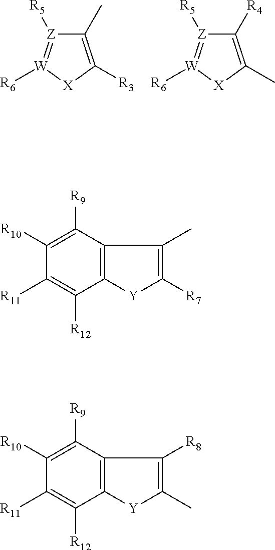

in which:
X and Y may be the same or different, and may represent an oxygen atom, a sulfur atom, an oxidized form of sulfur, a nitrogen atom or a selenium atom;
Z and W may be the same or different, and may represent a carbon or nitrogen atom;
the radicals R₃ to R₁₂ may be the same or different, and may represent a hydrogen, a linear or branched C₁-C₁₆ alkyl or alkoxy group, a halogen, a linear or branched, fluorinated or perfluorinated C₁-C₄ group, a carboxyl group, a C₁-C₁₆ alkylcarboxyl group, a C₁-C₁₆ mono- or dialkyl-amino group, a nitrile group; a phenyl group, a naphthalene group or a heterocycle (pyridine, quinoline, thiophene) may be substituted onto said radicals.

However, groups A and B must not both be equal to an indole type structure such as that below:

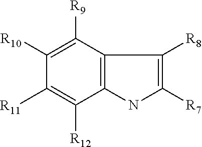

Groups A and B may be separated from the cycle by one or more double bonds.

In the positions ortho to the junction, between the double bond and the residues A and B, there must always be a group other than hydrogen, for example CH₃, CN, or COOEt, i.e. groups R₃ or R₅, R₄, R₇ and R₈ must be other than hydrogen.

An example that may be mentioned is the following compound that changes from colorless to red as follows, after irradiation at 404 nm-436 nm (return at 546 nm-578 nm):

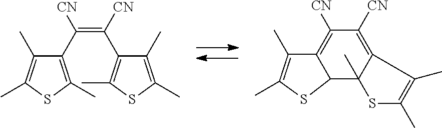

One example of a diarylethene is that which is blue in color in the developed state, sold under the trade name: DAE-MP by Yamada Chemical (Japan), with chemical name and formula: 1,2-bis(2-methyl-5-phenyl-3-thienyl)-3,3,4,4,5,5-hexafluorocyclopentene:

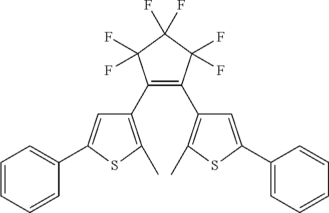

Another example of a diarylethene is that with a yellow color in the developed state, with formula:

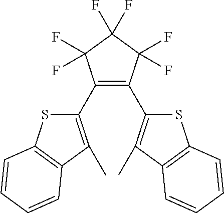

sold under the trade mark: DAE-2BT by YAMADA CHEMICALS

(Japan) and with chemical name: 1,2-bis(3-methylbenzo(b)thiophen-2-yl)perfluorocyclopentene.

Fulgides may be represented by the following formula:

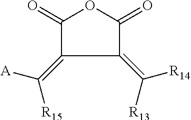

in which:
group A has the meaning given above;
groups R₁₃ to R₁₅ may be the same or different, and may represent a C₁-C₁₆ linear or branched alkyl group, or groups R₁₃ and R₁₄ may form a cycle containing 3 to 12 carbon atoms, such as a cyclopropane or an adamantylene.

### Other Thermally Stable Photochromic Agents

These photochromic compounds are compounds that change appearance, for example changing from a colorless or faintly colored form to a colored form via a mechanism controlled by light irradiation.

The mechanism may be direct, in the sense that light causes the appearance to change, for example by changing from the colorless form to the colored form. This applies, for example, with compounds carrying a photodegradable or photorelease function.

Preferably, compounds are used wherein the photodegradable or photorelease function is inert relative to keratinous material.

Preferably, a compound is used wherein the photodegradable or photorelease function is immobilized or carried by a polymer or other solid or bulk structure.

As an example, the 3-5-dimethoxybenzoin function and colored products as described in the article: C. P. McCoy et al., J. Am. Chem. Soc., 2007, 129, 9572, may be used.

The above mechanism may also be indirect, in the sense that light causes the compound, which is initially colorless, for example, to change into another form, then a third action transforms that modified, but still colorless, compound into a form with a different appearance, for example colored.

The mechanism is also indirect when a third action acts differently on the compound that has not been modified by light and on the modified compound and, for example, the unmodified compound is transformed and changes appearance, becoming colored, for example, while the modified compound conserves its appearance by colorless coupling.

As an example, it is possible to use the diazotype principle, which uses a diazonium salt compound and another aromatic compound that is capable of reacting by a coupling reaction. Irradiation destroys the diazonium salt (liberation of nitrogen). The diazonium compound that has not been irradiated then reacts by a simple jump in pH (in the presence of ammonia, for example) with the coupler to produce an azo compound. Under such circumstances, non-colored diazonium salt compounds are selected, such as an aromatic compound carrying the diazonium function but not carrying an amine or hydroxyl function on the cycle. The coupler may be a simple aromatic amine such as an aniline or phenol derivative.

Thus, the non-irradiated portions are developed, in accordance with the reaction:

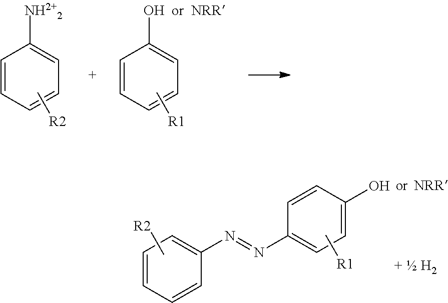

The photochromic agent or agents used may become thermally stable upon development or only after a specific action has been carried out, for example being brought into the presence of chemical compounds endowing them with the desired thermal stability.

The thermally stable photochromic composition may contain a total of 0.001% to 20% by weight of photochromic agent(s), in particular thermally stable photochromic agent(s).

The photochromic composition may also contain any solvent that is appropriate for cosmetic application, in particular selected from those mentioned in patent EP-A-0 938 887.

The composition may comprise the ingredients named in paragraphs [0029] to [0041] of EP-A1-0 938 887; the list is hereby incorporated by reference.

### Reduced Sensitivity Thermally Stable Photochromic Composition

The thermally stable photochromic composition may include at least one optical agent reducing its sensitivity to UV or near UV radiation.

The thermally stable photochromic composition may in particular comprise one or more optical agents in a quantity sufficient for its screening power F as defined below to be 2 or more, or even 5, 10, 15, or 20.

### Protocol for Measurement of Screening Power

A protocol similar to that used to determine the SPF is used, the difference being that the erythemal response of the skin is not taken into account.

The composition of screening power that is to be discerned is applied at 1.2 mg/cm² [milligram per square centimeter] to a sanded polymethyl methacrylate (PMMA) plate (without UV screen) measuring 5 cm [centimeter] by 5 cm, 3 mm [millimeter] in thickness, with a roughness of 4.5±1 μm, from EUROPLAST. The plates are pre-treated with a deposit of 10±1 mg of Vaseline 145B. The composition may be deposited in 14 dots of composition and spreading is carried out for 20 seconds using a finger, making zigzags and turning the plate by one fourth of a turn every 5 seconds.

After spreading, 0.6 mg/cm² of composition subsists. It is allowed to dry for 20 minutes (min), and is then spread again.

A spectroradiometer is used (for example a Labsphere UV transmittance analyzer with an integrating sphere), which measures diffuse transmittance from 290 mm to 400 mm. Each value for transmission T(λ) is recorded. T(λ) is the ratio, for a given irradiation wavelength λ, of the transmitted light energy over the incident light energy. 5 measurements are taken per plate (moving the plates) and the mean of these 5 measurements is taken. The operation is carried out on 5 plates. The means of the 5 measurements is taken.

The screening power F relative to solar UV radiation (290 nm to 400 nm) is given by the ratio of the following two integrals:

$$F = \frac{{\int }_{290 n m}^{400 n m} I ( λ ) d λ}{{\int }_{290 n m}^{400 n m} I ( λ ) T ( λ ) d λ}$$

where I(λ) is a function representing the occurrence of each wavelength of the solar spectrum. I(λ) is the same as that used to calculate the in vitro SPF in the publication COLIPA GUIDELINES Edition of 2007: A METHOD FOR THE IN VITRO DETERMINATION OF UVA PROTECTION PROVIDED BY SUNSCREEN PRODUCTS. If F=1, then the composition does not screen.

The term “act as a screen to radiation with wavelength λ” means that the optical agent attenuates radiation with a wavelength λ by at least an attenuation factor of 2, the measurement being carried out using an apparatus for measuring the absorption spectrum, restricting the irradiating light to a zone with a wavelength λ±10 nm. The ratio

$${F}_{λ - 10 , λ + 10} = \frac{{\int }_{λ - 10}^{λ + 10} I ( λ ) d λ}{{\int }_{λ + 10}^{λ + 10} I ( λ ) T ( λ ) d λ}$$

where I(λ) and T(λ) are as defined above, provides the attenuation factor at wavelength λ.

The thermally stable photochromic composition may have at least one range P of wavelengths in the interval λ₁ to λ₂ where the irradiation is less screened, the screening power in this range being a mean of F_{λ1,λ2}, where F/F_{λ1,λ2}>2, preferably F/F_{λ1,λ2}>5. The width of the range p may be less than 80 nm, preferably less than 40 nm.

F_{λ1,λ2} is defined by:

$$\frac{{\int }_{{λ}_{1}}^{{λ}_{2}} E ( λ ) d λ}{{\int }_{{λ}_{1}}^{{λ}_{2}} E ( λ ) T ( λ ) d λ}$$

$$\frac{{\int }_{λ 1}^{λ 2} I ( λ ) d λ}{{\int }_{λ 1}^{λ 2} I ( λ ) T ( λ ) d λ}$$

and measured as described above, replacing the limits 290 and 400 by λ₁ and λ₂, where λ₂>λ₁.

Where appropriate, the thermally stable photochromic composition may comprise a color-change colorant, for example a coloring agent of color that develops over time and if possibly slowly, for example DHA or polyphenols, which may slowly become colored in contact with air. The thermally stable photochromic composition may, for example, comprise a coloring agent that takes more than half an hour to become 90% developed. The advantage of such a coloring agent may be that it develops a screening power once the light-sensitive makeup look has been created in order, for example, to retard the degradation of the light-sensitive makeup patterns under the effect of ambient light.

### Second Layer Acting as Activator for an Optical Agent Present in the First Layer

In one implementation of the invention, a first layer is constituted by the thermally stable photochromic composition and contains an optical agent in the partially or completely deactivated form or as a precursor. This agent in the deactivated form or in the precursor state has not yet been activated sufficiently to protect the result of the light-sensitive makeup.

After the light-sensitive makeup, application of a second layer serves to activate the deactivated optical agent or bring the precursor into the form of an optical agent that is effective in forming a screen to the radiation developing the thermally stable photochromic composition.

As an example, in a precursor form in one of the layers, the optical agent may be a coloring agent from the porphyrin class, rendered more active by the presence in the other layer of a salt in solution, for example a zinc, iron, or magnesium salt.

### Galenical Forms

The thermally stable photochromic composition may be presented in various galenical forms depending on the applications and the nature of its constituents.

The thermally stable photochromic composition of the invention contains a cosmetically acceptable medium, i.e. a medium that is compatible with all keratinous materials such as the skin, the nails, the hair, the eyelashes and eyebrows, the mucous membranes and the semi-mucous membranes, and any other cutaneous zone of the body and face. In particular, said medium may comprise or be in the form of a suspension, a dispersion, a solution in a solvent or hydroalcoholic medium, optionally thickened or gelled; an oil-in-water emulsion, a water-in-oil emulsion, or a multiple emulsion; a gel or a foam; a gel emulsion; a spray; a loose, compact, or cast powder; an anhydrous paste; or a film, inter alia.

### Treated Zones

Any part of the body that is normally made up may receive light-sensitive makeup in accordance with the invention, for example the nails, eyelashes, hair, skin and in particular that of the face, for example the cheeks, the forehead, the lips or eye contour, the neck, the chest or the legs.

It is also possible to treat parts of the body that are rarely made up, such as the ears, the hands, or the teeth. These zones have complex shapes that do not assist in making the application of conventional makeup products easy. Light-sensitive makeup enables esthetic results to be obtained, despite their complex shapes.

Light-sensitive makeup may be used to camouflage a skin blemish.

Light-sensitive makeup may optionally repeat a pattern from clothing or an accessory worn by the user, for example a pattern on a piece of jewelry, a purse, eyeglasses, shoes, a piece of furniture, a personal digital assistant (PDA), or a cell phone.

Where appropriate, sale of such clothing or such an accessory may be accompanied by the provision of a file or an internet link that allows a light-sensitive makeup look to be created that coordinates with the accessory or clothing.

The file or internet link may provide access to the data necessary for producing an image that has been designed or selected in order to coordinate with the clothing or accessory. As an example, it may repeat all or part of the patterns or it may complete them.

### DESCRIPTION OF THE FIGURES

FIG. 1 is a diagrammatic and fragmentary view of an example of a system for processing light-sensitive makeup produced in accordance with the invention;

FIG. 2 illustrates how the zone to be treated is held during light irradiation;

FIG. 2a illustrates the formation of a pattern within a pixellated image;

FIGS. 3 to 6 are diagrammatic and partial views of variations of the irradiator;

FIGS. 7 to 10 and 10A illustrate different examples of addressable matrix imagers using several technologies;

FIGS. 11 to 13 show examples of light-sensitive makeup;

FIGS. 14A to 14C and 15A to 15C show examples of the progress of light-sensitive makeup;

FIG. 16 represents an example of a packaging device enabling the composition to be developed before it is applied; and

FIG. 17 represents an example of makeup as a function of an image projected on the zone to be treated.

### MODES OF APPLICATION

The or each composition suitable for use in implementing the invention may be applied in the form of a powder, fluid, spray, or film. The fluid may have different rheologies. It may, for example, be a block of product that spreads when rubbed onto keratinous material, or it may be a liquid.

The layer of thermally stable photochromic or optionally photoprotective composition, and rather the photoprotective composition layer, may be applied in the form of a dry or nearly dry powder.

The or each composition may optionally be in the form of a pre-formed film.

Preferably, when using a multilayer application, the second layer is preferably applied without deteriorating the first layer that has already been applied. To this end, it may be preferred to apply the composition intended to form the second layer by spraying.

Application may also be carried out using a printer, for example an inkjet printer, using an apparatus that is brought into contact with the zone to be treated, and optionally that is moved over it. When one or more layers are sprayed, any spray technique may be used, for example spraying by means of a propellant gas, an airbrush, or electrostatic or piezoelectric spraying. The application may also be carried out by transfer, using a support sheet carrying at least one of the compositions, or even the various layers to be formed. The transfer may be accomplished by pressure, by heat, and/or by using a solvent deposited on the support sheet and/or keratinous material that are to be treated in accordance with the invention.

The application may be carried out manually or in an automatic manner, e.g. using a manipulator arm.

Each layer may be applied after drying any layer that precedes it.

The application may be made using applicators, possibly single use applicators, comprising an application member loaded with composition to be applied.

The compositions may be applied at a point of sale, in a beauty salon, or in the home, inter alia.

Each composition may be packaged before use into any suitable receptacle.

The compositions, and in particular the thermally stable photochromic composition, may be applied after verifying that the intensity of the ambient light is not prejudicial to the quality of the light-sensitive makeup look. Verification may be carried out using a warning device that alerts the user if the ambient light contains UV or near UV radiation that is too strong, for example with a flux of 0.1 mW/cm² or 0.5 mW/cm², the threshold being adjustable if necessary. Such a device may be autonomous or integrated with a packaging device or a device for application of the thermally stable photochromic composition, or even a packaging device or device for application of a photoprotective composition.

Where appropriate, the information delivered by the warning device may also be useful for selecting a thermally stable photochromic composition, a photoprotective composition, and/or a composition serving as a base layer amongst a plurality of compositions, depending on the level of the UV radiation.

As mentioned above, where appropriate, the thermally stable photochromic composition may be applied in the completely developed state or in the non-developed state.

The thermally stable photochromic composition and the composition intended to form the coating layer or the base layer or the photoprotective layer may be in a variety of forms, for example creams, gels, liquids, in the form of compositions to be spread with the hand or using an applicator, for example a roll-on.

The composition may be applied by moving a block of composition in contact with the keratinous material, such as a lipstick, for example. Further, the composition may be applied by spraying using an aerosol can, a pump bottle, or an electrostatic, piezoelectric, or airbrush spray device.

The composition may be in dry form, such as a powder, which may be applied with a brush or paintbrush if required.

When the photochromic composition is intended to be applied in the developed state, it may be contained in a packaging and application device as shown in FIG. 16, comprising a recipient 1000 containing the composition, a light source 1010 emitting in the UV, for example, to develop the composition, and applicator means, for example an applicator paintbrush 1020.

### System for Processing Light-Sensitive Makeup

The light-sensitive makeup look may be created using a system 1 for processing light-sensitive makeup comprising an irradiator 3 comprising at least one light source 2, as may be seen diagrammatically in FIG. 1. Depending on circumstances, the imager may serve to develop the thermally stable photochromic composition, for example by causing the photochromic agent or agents it contains to pass from a non-developed state to a developed state and/or by serving to erase the photochromic agent or agents contained in the thermally stable photochromic composition by causing them to pass from a developed state to a non-developed state. When the composition is initially in the non-developed state, the irradiator may, for example, emit in the UV or near UV when the thermally stable photochromic composition may be developed by UV or near UV radiation.

The image may be defined by a mask or a negative disposed in the path of the light traveling to the zone to be treated, and optionally in contact with the zone to be treated.

The irradiator preferably comprises one or more imagers 4 for forming at least one image at a distance on the zone to be treated Z.

In a simplified version, the imager or imagers may use a mask or a negative and optics for projection on the zone to be treated. The negative may be a UV negative that allows an image to be formed in UV or near UV light.

The source 2 may comprise any type of luminous element, for example an incandescent lamp, a halogen lamp, a discharge lamp, and/or an electroluminescent element, in particular one or more light-emitting diodes (LEDs), organic LEDs (OLEDs) or other electroluminescent technologies.

As is described in detail below, the irradiator 3 may comprise a plurality of sources in order to emit in the UV or near UV and also in the visible, in particular the near-UV visible.

Advantageously, the irradiator comprising the light source or sources and the imager or imagers are capable of emitting selectively in the UV or near UV and in the visible.

Changing a projection of an image in visible light to a projection of an image in UV light may be accomplished by changing the source by using a movable mirror, by using a semi-transparent surface, by adding or removing a filter, and/or by using a frequency doubler or tripler, for example.

As is described in detail below, using visible light enables the image projected on the zone to be treated to be seen before carrying out development and/or causing the light-sensitive makeup to be removed from a zone that has already been at least partially developed, when the photochromic agent or agents allow this.

As may be seen in FIG. 1, the system for processing light-sensitive makeup may comprise a computer 10 that may be associated with a user interface 11 comprising, for example, a keyboard, a mouse, a touch screen, a voice recognition engine, a graphics tablet, a joystick, and/or a touch pad; this list is not limiting.

The computer 10 may comprise a microcomputer and, more generally, any computing means, analog and/or digital, produced using microcontrollers, microprocessors, and/or programmable logic arrays, for example.

The computer 10 may be produced in the form of one or more appliances, and when an electronic imager is used, where appropriate the imager may carry out all or some of the computations. The computer 10 may also be associated with a display means 12 that is, for example, a color screen, for example of the LCD, plasma, OLED or cathode ray type, optionally a touch screen. As described below, this display means 12 may be used to display the treated zone during treatment, enabling the treatment to be controlled and/or a simulation to be displayed.

The computer 10 may also be associated with data storage means 13, for example a hard disk, a magnetic tape, an optical disk, and/or flash memory, the data storage means possibly being integrated into the computer 10, the irradiator 3, and/or at least partially remote in an external data storage system.

The computer 10 may be associated with a network interface 14 that serves, for example, to download data pertaining to light-sensitive makeup or to cause data concerning the light-sensitive makeup that is being applied or has been applied to be transmitted to third parties or to a server.

Where appropriate, the computer 10 may advantageously control the source or sources producing the light used to form the image, so that the image is formed with a pre-defined illuminant.

The imager is advantageously an electronic addressable matrix imager as is described below, to which the computer may send data in order to cause a pre-defined image to be projected onto the zone Z to be treated.

The system for processing light-sensitive makeup may be provided with at least one optical and/or electronic system enabling the image to be focused manually or automatically, and advantageously with means to prevent movements.

In order to keep the zone being treated still relative to the irradiator, the system for processing light-sensitive makeup may comprise means for keeping the person still, meaning that movement and fuzzy results may be avoided.

When the image is formed on a face, the system for processing light-sensitive makeup may be configured to detect that the face is relaxed, and to control the imager as a function of that detection.

The system 1 for processing light-sensitive makeup may comprise a flat portion or it may follow the shape of a part of the body and that part may be placed against the zone to be treated.

In a variation or in addition, the system for processing light-sensitive makeup may comprise a system for rectifying movements, e.g. of the same type as those used to stabilize images in still or motion picture cameras.

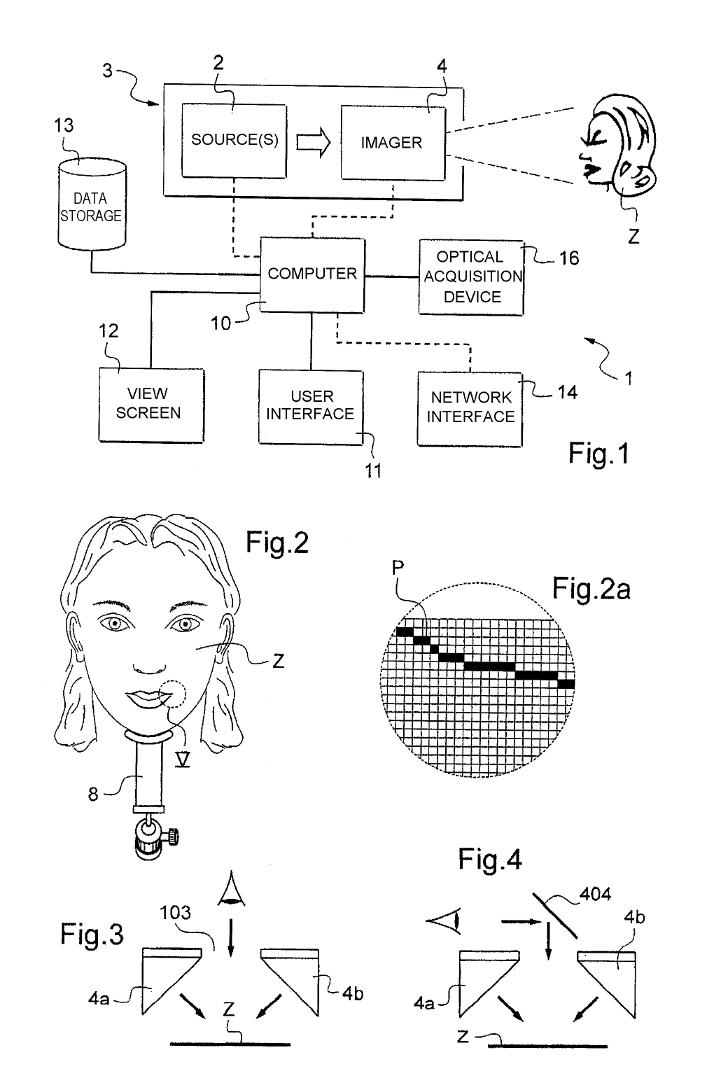

When the face is treated, the system for processing light-sensitive makeup may comprise a means 8 for immobilizing the individual to be treated, in the form of a chin rest, as may be seen in FIG. 2.

In a variation, the irradiator 3 is portable and may be fastened on a mount carried by the individual to be treated, in order to illuminate the face, for example.

The system 1 for processing light-sensitive makeup may comprise an optical acquisition device 16 that, in one implementation of the invention, may transmit data to the computer 10 so as to cause it to propose a light-sensitive makeup look and/or so as to control the production thereof, as is described in detail below. The optical acquisition device 16 may advantageously have a viewing axis that is substantially parallel to the direction of projection of the image. The optical acquisition device may be monopixel or multipixel, and it may receive the light emitted by the imager directly or it may receive the light reflected by the zone Z to be treated.

The optional optical acquisition device, the irradiator, the optional computer, and the optional view screen may be produced in the form of separate elements or they may be integrated in the same casing. The irradiator and the optical acquisition device may advantageously be integrated in the same casing or they may be fastened together by other means.

The view screen may be fastened to the back of the casing of the irradiator or integrated into the casing. Where appropriate, the optical acquisition device comprises an internal lighting means for close-up acquisition.

The casing of the irradiator may also be movable and may be applied to the skin, for example, or it may be held in the hand. In one implementation of the invention, the casing of the irradiator may, for example, be placed on a table. The face may then be moved close to be placed on the casing, e.g. by leaning.

The system for processing light-sensitive makeup may be provided with means for detecting opening or closing of the eyes and/or the mouth in order to stop or not begin irradiation if the eyes and/or the mouth are open. The optical acquisition device 16 may provide an image that is analyzed for this purpose by the computer 10.

When the image is formed on a face, the system for processing light-sensitive makeup may be configured to identify the face and the imager may be controlled as a function at least of this identification.

The system for processing light-sensitive makeup is advantageously designed to allow a user to evaluate progress of the light-sensitive makeup, visually or otherwise.

To this end, the system for processing light-sensitive makeup may include a window allowing direct viewing of the zone being treated during irradiation, this viewing being carried out, where appropriate, through a UV screen. In order to allow room for direct viewing, the light may be emitted from an offset position and optical fibers or at least one mirror or a prism may be used to direct the light and focus the radiation on the zone to be treated.

FIG. 3 is a diagram showing part of a system for processing light-sensitive makeup that comprises two imagers 4a and 4b, respectively emitting ultraviolet light and visible light, towards the zone Z to be treated. A window 403 is provided between these imagers 4a and 4b, to allow observation of the zone Z during treatment.

The viewing zone may also be offset using a mirror 404 or any other optical system, for example optical fibers or a prism, as may be seen in FIG. 4.

In the presence of an optical acquisition device such as a digital (motion picture or still) camera, the treated zone Z may be viewed on a screen that may be placed on the irradiator or that may be offset.

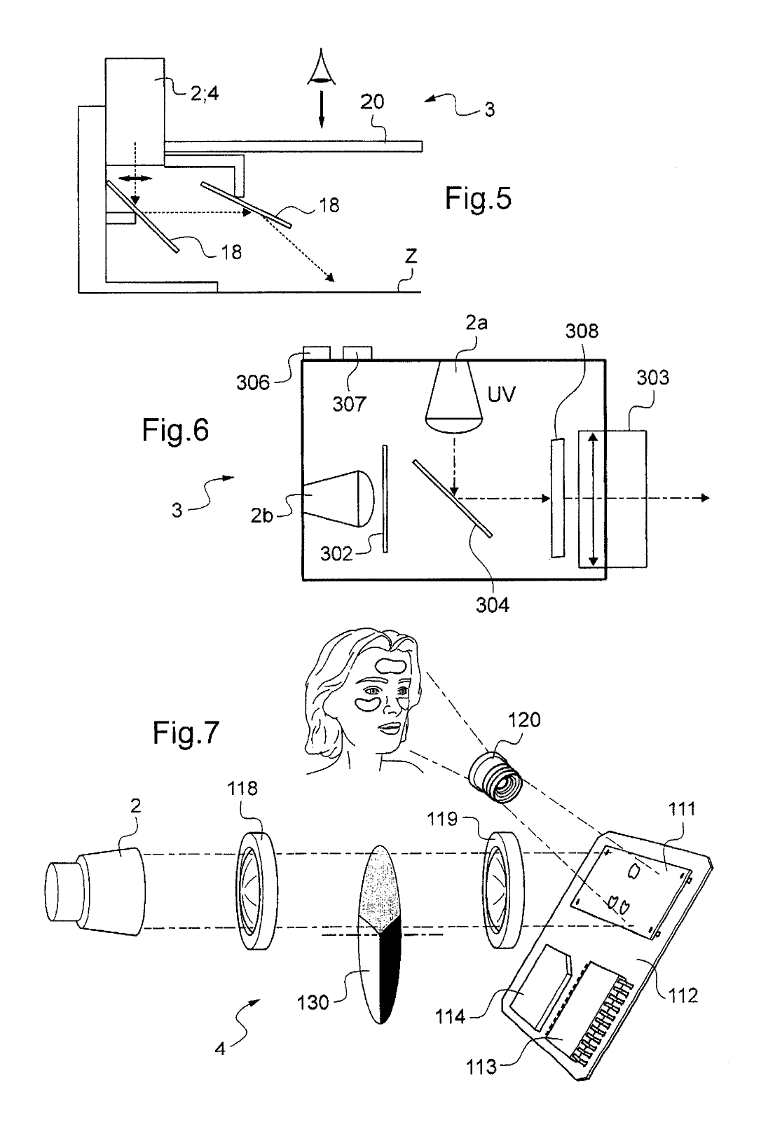

FIG. 5 illustrates the possibility of producing the irradiator 3 by offsetting the light beam directed towards the zone Z to be treated by using mirrors 18, which allows the user to observe the treated zone Z through a window 20 of the irradiator.

FIG. 6 illustrates the possibility of producing the irradiator 3 with two light sources 2a and 2b respectively emitting in the UV and in the visible. The irradiator shown in FIG. 6 comprises a color filter 302, for example a green filter, placed in front of the source 2b, adjustable collimation optics 303, and a movable mirror 304. The irradiator 3 in this example allows a negative 308 to be placed in the optical path. The adjustable collimation optics 303 causes the image of the negative to appear at a certain distance from the optical outlet of the irradiator 3, for example at about twenty centimeters.

The irradiator 3 is provided with two switches 306 and 307. The first actuates the sources 2a and 2b. The movable mirror is disposed so that only visible light is directed towards the optical outlet for a given position of the second switch. Actuating the switch moves the movable mirror, for example by activating a micromotor or an electromagnet, and the UV irradiation is then directed towards the negative 308.

As mentioned above, the system for processing light-sensitive makeup advantageously comprises an electronic addressable matrix imager.

### Electronic Addressable Matrix Imager

By way of example, an addressable matrix imager is suitable for projecting a pixellated image with a resolution of more than 10 by 10 pixels, preferably more than 10 by 100 pixels.

When the imager is an electronic addressable matrix imager, the image formed on the zone to be treated is formed by pixels that are on or off, optionally each at a pre-defined gray level. As an example, FIG. 2A shows a detail of FIG. 2 where the light-sensitive makeup P that is produced consists of a lip outline. FIG. 2A shows the placement of the various pixels of the projected image; only the pixels corresponding to the outline to be produced have been switched on. Development of the thermally stable photochromic composition matches the states of the pixels.

The light leaving the addressable matrix imager may be monochromatic or multichromatic; preferably, the addressable matrix imager is capable of selectively emitting in the UV or near UV and also in the visible beyond the near UV, the light emitted in the visible possibly being white light or a colored light, optionally monochromatic light.

The computer 10 may determine the digital image on the basis of which the electronic imager is controlled, in particular the gray level of each pixel, and optionally also the dominant wavelength of the light at each pixel.

Several technologies may be used to produce the addressable matrix imager.

It is possible to use the technology known as DLP (digital light processing) invented by TEXAS INSTRUMENTS, which uses a DMD (digital micromirror device) chip composed of thousands of micromirrors of orientations that are individually controllable by using an electric pulse, and depending on their orientation they may optionally reflect an incident beam of light in order to transmit or not transmit it to the optical outlet of the imager. The image to be projected is formed on the matrix of mirrors. The gray level in each pixel (for example 256 levels) may be controlled by adjustment of the mark space ratio.

FIG. 7 shows an example of an electronic imager 4 produced using this technology, using a DMD chip with reference numeral 111. The chip may be fastened on a platen 112 that may also include a processor 113 to control the chip, and also an optional memory 114. In the example shown, the chip is shown on the same platen as the processor 113 and the memory 114, but these items may be placed elsewhere.

The imager 4 shown in FIG. 7 receives light from a source 2 that may be a source capable of emitting both in the UV and/or in the visible or a source capable of emitting selectively in the visible or in the UV.

The source 2 may be a halogen lamp emitting in the UV and visible spectra, a discharge lamp, or one or more LEDs that are capable of emitting in the UV and of emitting white light, or light of a given color, for example.

As illustrated, the imager 4 may include optics 118, 119, and 120 respectively to condense the light, focus it on the DMD chip, and bring it to the zone to be treated.

When the source 2 has an emission spectrum in both the UV and in the visible, as illustrated, the imager 4 may have a filter wheel 130 that intersects the light beam between the condensation optics 118 and the focusing optics 119, for example. Depending on the position of the filter wheel 130, the chip receives UV or visible light that is then directed towards the optical outlet. Thus, it is possible to form an image on the zone to be treated selectively from visible light and/or UV light.

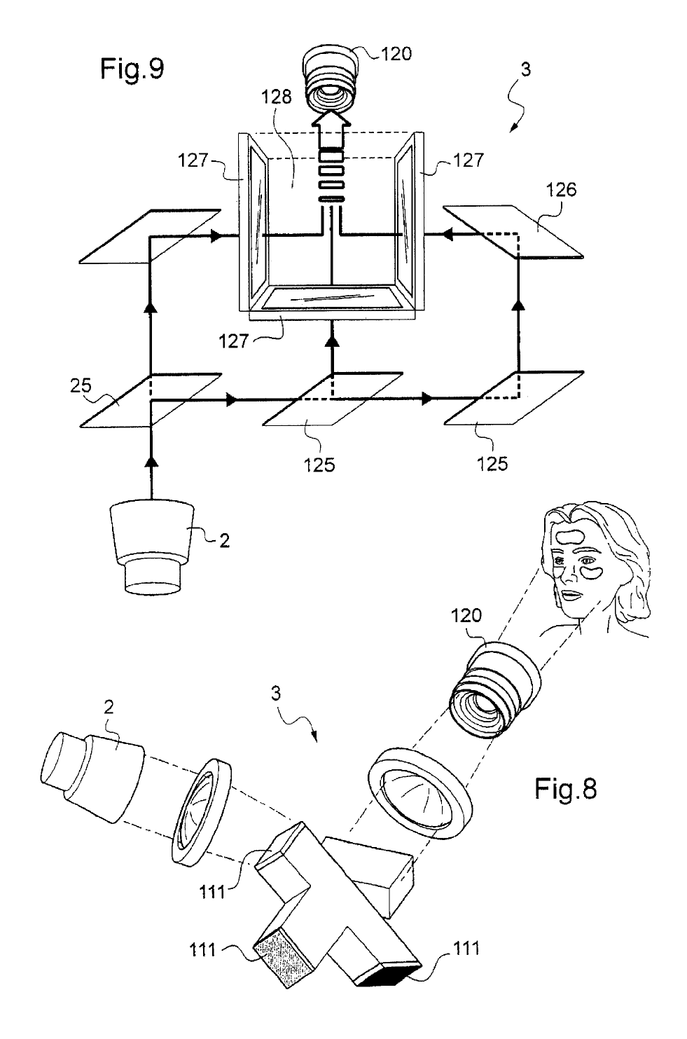

The irradiator in the variation of FIG. 8 uses a plurality of DMD chips fastened on a prism that divides the incident light from the source 2 into at least two beams with different dominant wavelengths, for example respectively in the UV or near UV and in the visible.

The light beams reflected by the DMD chips are projected towards the zone to be treated.

By controlling the DMD chip associated with the UV or near UV beam and the chip associated with the beam of visible light, either a beam of UV light or of visible light or possibly of both at once may be projected onto the zone to be treated; when development takes place relatively slowly, this may be useful in order to be able to visually monitor the proper positioning of the light acting to carry out the development.

The irradiator may also use liquid crystal display (LCD) technology.

In the example of FIG. 9, the source 2 is directed onto dichroic mirrors 125 that generate at least two light beams with different dominant wavelengths, one of said beams having a dominant wavelength in the UV or near UV and the other in the visible, for example.

The beams are directed by the mirrors 125 and 126 towards the LCD matrix screens 127 on which the image to be projected is formed, producing monochrome images directed towards a system of prisms 128, enabling the image to be sent via the projection optics 120 to the surface to be treated. Depending on the degree of opacity of the screens 127, the light emitted is in the visible region or in the UV region.

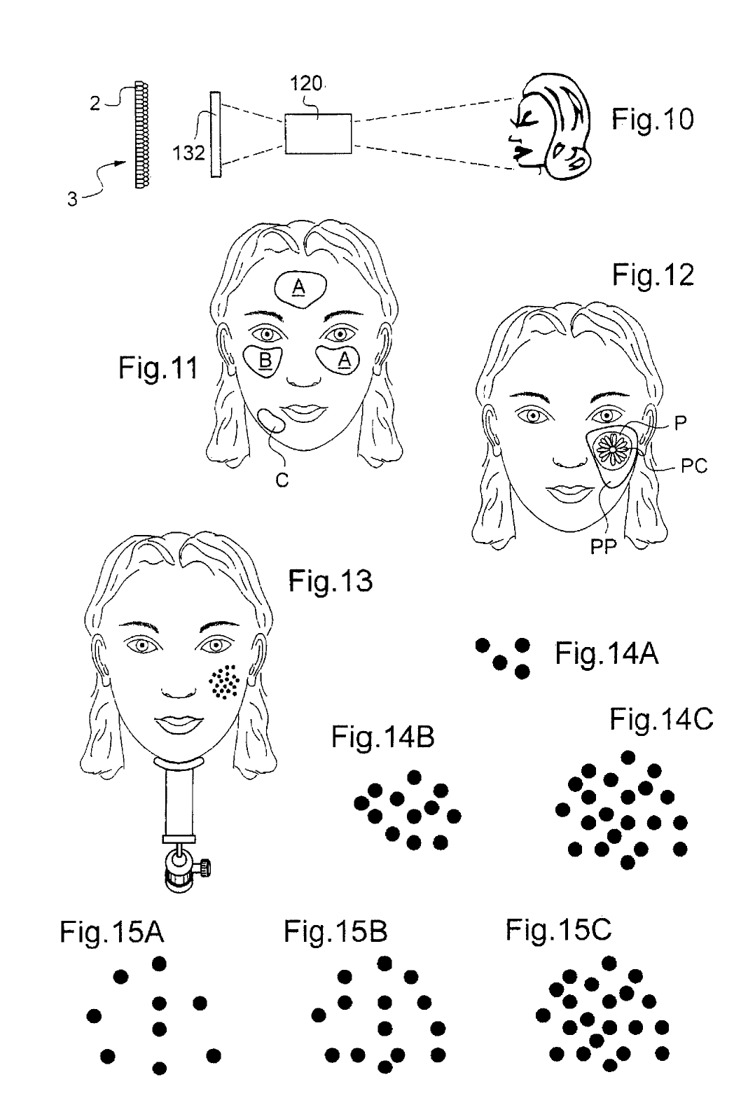

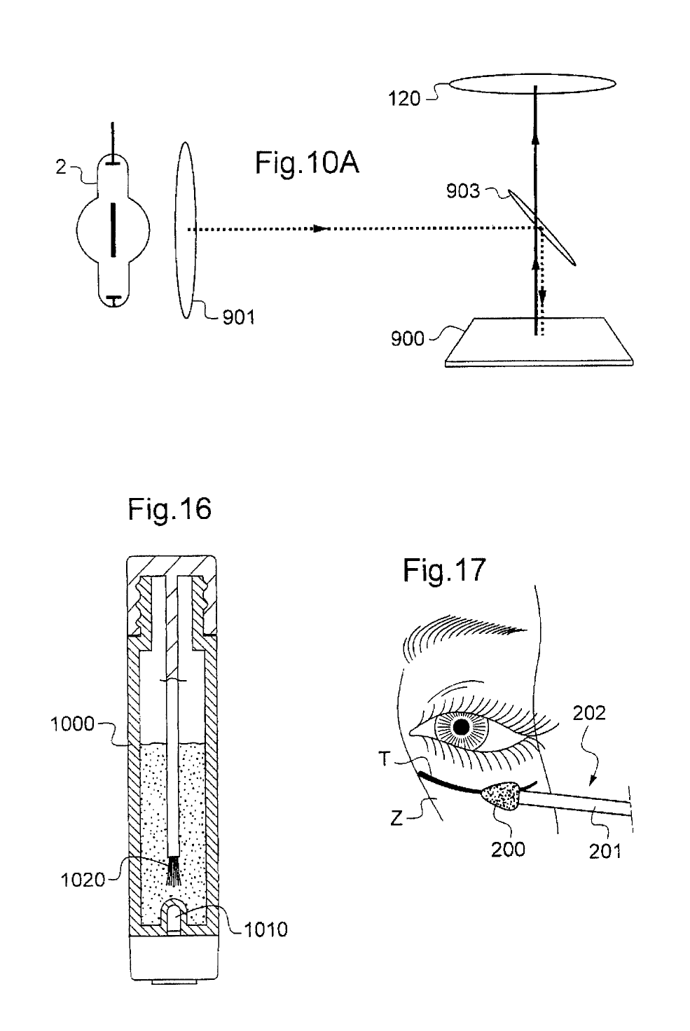

The irradiator 3 illustrated in FIG. 10 comprises an LCD matrix screen 132 and a source 2 that illuminates the screen 132. The image formed thereon is projected onto the zone to be treated by means of the projection optics 120. By way of example, the source 2 is capable of selectively emitting in the UV or in the visible.

In a variation, the screen 132 may replace the negative 308 of the example of FIG. 6.

The projection system may also be based on liquid crystal on silicon (LCOS) technology. LCD technology is termed transmissive because the light passes through an LCD screen, while DLP technology is termed reflective since the light is reflected by the micromirrors of the DMD chip. In LCOS technology, the mirrors of the DMD chips are replaced by a reflective surface covered with a layer of liquid crystals that may be switched between a light-passing state and a light-blocking state. By modulating the frequency at which the liquid crystals are turned on and off, the gray level of a pixel may be varied.

By way of example, the arrangements illustrated in FIGS. 7 and 8 may be used, replacing the DMD chips with LCOS chips.

FIG. 10A shows an LCOS chip irradiator. A system of lenses 901 may be disposed between the source 2, e.g. a UV lamp, and a semi-transparent mirror 903. This reflects light from the source to the chip 900. The chip reflects the light again to a focusing system 120 that projects the pixellated image onto the zone to be treated.

In general, the image delivered by the addressable matrix imager comprises a matrix of pixels of gray levels that are individually addressable, each gray level, for example, being coded into at least 4 bits, preferably 8 bits. The light associated with each pixel may also be coded where appropriate.

The image to be projected may be supplied to the electronic imager in the form of a video signal complying with the VGA, SVGA, composite, HDML, SVIDEO, YC_{B}C_{R}, optical video signal, or other standard, or in the form of a video or digital image file, e.g. a .jpeg, .pdf, .ppt, etc file. When these images are not monochrome, a pre-defined color on the image in the file may control the quantity of UV or near UV, for example.

The electronic imager is advantageously produced so as to be able to change the nature of the light emitted without changing the image; as an example, the pixels of the image retain their gray levels and only the emission spectrum of the source used upstream changes. This enables an image to be visualized on the zone to be treated and then the zone may be developed, simply by modifying the emission spectrum of the source.

The imager may be used to project visible light in order to selectively erase one or more photochromic agents and to create a light-sensitive makeup look from a thermally stable photochromic composition in the developed state. The imager may then be a conventional video projector, for example.

### Choice of Projected Image

In particular when using an electronic addressable imager, the system for processing light-sensitive makeup is preferably provided with a means for selecting the projected image. This may be accomplished by selecting from a library of images, possibly by displaying a succession of images from said library and the user selecting a displayed image. The images may be stored in the digital or photographic form, for example in the data storage means. The image library may be included in the system for processing light-sensitive makeup, or it may be downloaded.

In one implementation of the invention, a tailored image is used starting from the individual intended to receive the light-sensitive makeup, or from a model such as a celebrity or an individual of given style, the images possibly being derived from made-up or non made-up people. It is also possible to use images derived from drawings, paintings, sketches, or caricatures to generate the projected image.

The computer may have in its memory or may download at least one pictorial model in the form of lines or brush strokes, or even a single point or a series of lines, strokes, or points.

The image formed may be determined automatically as a function of the acquired image. This enables the projected image to be adapted to the morphology and/or to the color of the face.

Starting from the position of the captured face, the computer may correctly position the image intended to create the light-sensitive makeup look.

The system for processing light-sensitive makeup may thus be used in a method comprising the steps consisting in:
acquiring at least one image of the zone of the subject to be made up;
controlling the imager as a function of the acquired image.

As an example, the image may be acquired using an optical acquisition device 16 that may be adapted to capture all or a portion of the face or any other treated zone of the body.

As an example, in order to create a light-sensitive makeup look on the upper eyelids, it is possible to carry out the following steps:
capturing the image of the face, and deducing the eyelid zone therefrom;
once the thermally stable photochromic composition has been applied to the eyelid zone, irradiating the pictorial model to be produced at the region of the eyelid zone; hence, the resulting light-sensitive makeup is formed at the correct location. Irradiation at the position of the zone to be treated may be carried out by lighting only the corresponding pixels, in a situation where the image is capable of covering a much more extensive zone when all of the pixels are lit. In order to benefit from better resolution, the treated zone may, for example, involve at least ⅔ of the total number of pixels of the image.

The computer may also modify the shape of the pictorial model in order to adapt it to the shape of the face. Thus, for example, if it is desired to make up the lips, the following steps may be carried out:
capturing the image of the face, and deducing the zone of the lips therefrom;
comparing the shape of the lips with the pictorial model to be reproduced;
modifying the pictorial model so that it is inscribed within the shape of the lips;
once a thermally stable photochromic composition has been applied to the lips, irradiating them to produce the modified pictorial model. This method may also be applied to other regions of the body.

Thus, the system for processing light-sensitive makeup may be provided with the following four functions:
capturing the image of the face or any other region of the body to be treated;
locking the position of the pictorial model to be produced on the portion of the face of body that is to receive it, by analyzing the image of the face or any other part to be treated;
optionally, modifying the shape of the pictorial model to adapt it to the shape of the face;
controlling projection of the image intended to produce the light-sensitive makeup.

The system for processing light-sensitive makeup may comprise means for acquiring the 3D shape of the face. The system for processing light-sensitive makeup may comprise an optical acquisition device that is adapted to detect relief, by projecting fringes, for example, and/or it may be adapted to detect shine.

In one implementation of the invention, the pictorial model used is determined automatically. This choice may be made in a random manner or by using programmed logic that uses rules to optimize the appearance of the face, for example to fit in with a color harmony scheme or a natural harmony scheme for the face. Thus, for example, for a white-skinned face, it is possible to produce freckles.

The choice may also be made by applying logic to re-establish symmetry for faces with asymmetries and/or by applying light and shade to cause a face that is too angular to be rendered rounder or vice versa, or to correct natural or unattractive proportions.

In one implementation of the invention, the system for processing light-sensitive makeup proposes a plurality of pictorial models, leaving the user at liberty to select one. These proposals may be expressed graphically, for example by display on a screen. The proposed pictorial model may be superimposed on an image of the subject intended to receive the light-sensitive makeup, or the model may be displayed on a screen describing the face by a diagram. Any interface that allows the user to select a pictorial model may be used. As an example, the description of a pictorial model proposed to the user may be given verbally by describing the actions that the system for processing light-sensitive makeup proposes to carry out.

The system for processing light-sensitive makeup may be configured to automatically detect a skin blemish on the zone to be treated and the imager may be controlled as a function of the nature of the detected blemish.

The system for processing light-sensitive makeup may be provided with particular recognition functions intended, for example, to recognize blemishes, for example:
spots, blackheads, pimples, strawberry spots, blotches;
wrinkles, cracks, stretch marks, veins;
raised or recessed portions in relief such as scars;
asymmetries;
desquamation;
matt or shiny skin;
hairs.

The blemishes may be detected by image analysis and/or relief analysis. The image analysis may be 3D image analysis. The image analysis may include analysis of color and/or shine.

The system for processing light-sensitive makeup may also be provided with functions that allow a pictorial model to be computed or selected for the purpose of limiting the visibility of said blemishes. Examples of these pictorial models that may be mentioned are those intended to blur some portions that are detected as having blemishes and those intended to alter the outlines of certain parts, especially scars or asymmetries.

In another implementation of the invention, the user or a third person may define the pictorial model to be produced. Thus, the user or the third person may transmit commands that are interpreted by the system for processing light-sensitive makeup. These commands may be graphical and the system for processing light-sensitive makeup may comprise a man-machine interface of the touch screen type. The user transmits makeup orders by designating, on the image of the face or on a diagram of the face, the zones on which a makeup line is to be produced. The system for processing light-sensitive makeup may be configured to interpret the instructions from the user, to adapt them to the topography of the face, and then to create the light-sensitive makeup look.

The commands may be descriptions, for example “fill the lip zone with red” or they may be intuitive, for example “eyelid makeup”. The system for processing light-sensitive makeup will then act, in a conventional or a specifically programmed manner, to interpret the default pictorial model that is to be used.

The commands may be programmed and the programs may be personalized.

The person who selects from the proposed pictorial models or who determines the pictorial models to be produced may be the person who is being made up or some other person, such as a professional makeup artist. Selection or production of the pictorial models may be made at the location where the light-sensitive makeup look is being created, or remotely. When acting remotely, the system for processing light-sensitive makeup may be provided with communications means enabling the image of the zone for treatment to be communicated, for example the above-mentioned network interface 14.

The system for processing light-sensitive makeup may optionally be provided with a means for capturing makeup looks from magazines or other media and of making pictorial models from them that could then be reproduced on the zone to be treated, for example a scanner or an RFID chip reader, the chip containing the description of the makeup look or an internet link allowing it to be downloaded. This chip could be contained in packaging containing the composition or compositions to be used to create the light-sensitive makeup look or be contained in an article of clothing or other accessory with a particular pattern, which could be reproduced with light-sensitive makeup.

The system for processing light-sensitive makeup may be configured to display a succession of many sorts of pictorial model, in the form of simulations, in order to allow a person to select the model to be reproduced from among them.

The system for processing light-sensitive makeup may offer the possibility of rapidly trying out many sorts of models, directly on the face. Thus, the person may find out on a real version whether the models will suit him or her. These models could be images projected in visible light onto the face, which do not develop the thermally stable photochromic composition, or light-sensitive makeup looks created using a thermally stable photochromic composition that is also erasable, for example by irradiation with visible light.

The system for processing light-sensitive makeup may advantageously have several pre-recorded models in its memory in the storage means 13 and may memorize the pictorial models that it has been able to create. In this manner, the user may use or exchange the recorded pictorial models.

In one implementation of the invention, once a pictorial model has been selected, adaptation of the pictorial model to the topography of the face of the person and creation of the light-sensitive makeup by projecting the image are carried out automatically. The time interval separating capture of the face and production of the image may be rendered relatively short, for example less than one second.

In another implementation of the invention, the person receiving the light-sensitive makeup or a third person may intervene while the operations are being carried out. Creating the makeup may then be slower than before. The system for processing light-sensitive makeup may be configured so as to allow the person or the third person to view the progress of the light-sensitive makeup, for example on the screen 12, in order to slow down or stop its progress.

The system for processing light-sensitive makeup may optionally regularly recapture the face in order to re-commence the operations of locking and adapting the pictorial model to the face, thereby eliminating any problems that might be caused by movement of the person during irradiation intended to develop the thermally stable photochromic composition.

It is possible to create several partial light-sensitive makeup looks in succession. Thus, during the course of creating the light-sensitive makeup look, each pictorial model may be determined, its effect may be estimated by eye, then the next pictorial model may be selected and so on, thereby progressively constructing the light-sensitive makeup.

As mentioned above, the system for processing light-sensitive makeup may be configured to evaluate the pictorial models that are the most adapted to a face or part of a face by means of one or more specialized programs. Thus, the light-sensitive makeup look may be created by producing a first pictorial model, then by evaluating the face a second time to deduce therefrom the new pictorial model to be produced, and so on.

It is possible to treat one portion of the face in a semi-automatic manner and another portion in an automatic manner. It is also possible to treat a portion of the face in an automatic manner up to a certain point, then to continue the light-sensitive makeup in a semi-automatic manner, or vice versa.

The system for processing light-sensitive makeup may be configured to take an image, for example using the above-mentioned optical acquisition device, optionally extract a portion corresponding to the zone to be treated, and where appropriate to rectify this image to thereby improve the result once projected.

The system for processing light-sensitive makeup used is preferably configured to allow the user, starting from an image projected on the face or any other zone to be treated, to rectify the shape, for example by enlarging, or shrinking in one or two dimensions. The modifications may also be more complex. Thus, for example, it is possible to rectify a portion of the image, stretch a particular zone, change the size of the lines, etc. For this, it is possible to use the tools normally present in software for producing and editing images, such as Photoshop® for example. Where appropriate, the image may be edited by feedback via the optical acquisition device; the computer will know the result of the projected image and automatically modify it until the desired result is obtained by means of the system for processing light-sensitive makeup program executing a loop.

### Creating Light-Sensitive Makeup Progressively

The light-sensitive makeup may be created by carrying out the steps consisting in:
applying a thermally stable photochromic composition to a zone to be treated;
irradiating the zone with light selected either to progressively develop the thermally stable photochromic composition, or else to progressively erase the thermally stable photochromic composition;
interrupting and/or modifying the characteristics of the irradiation when the desired appearance is achieved, this appearance corresponding, for example, either to partial development of the thermally stable photochromic compound, or else to partial erasure of the thermally stable photochromic composition.

Thus, it is easier to obtain makeup results of the intended intensity. During progressive illumination, the user and/or the system for processing light-sensitive makeup may monitor the progress of the light-sensitive makeup and may stop it changing once the desired result is achieved.

Similarly, if the thermally stable photochromic composition allows it, editing may be carried out to further refine the light-sensitive makeup, either at the time of light-sensitive making up or later on.

Irradiation may be interrupted then recommenced at least once.

The dominant wavelength and/or intensity of the irradiation may be modified before the desired appearance is achieved. As an example, by modifying the intensity of the irradiation, the rate of development or erasure of the thermally stable photochromic composition may be changed. By adjusting the dominant wavelength, it is possible to adjust the irradiation energy and/or the effect exerted on the photochromic agents.

The whole of the image may be treated progressively, but it is also possible to treat the image portion-by-portion in a progressive manner, for example in an automatic, programmed, or programmable manner.

The light-sensitive makeup may be used to create several patterns in succession. At least one pattern that has achieved the desired appearance may stop being irradiated while at least one other pattern is still being irradiated. The patterns are, for example, freckles, which may be created in succession.

Thus, a specific program may be executed to produce freckles, as can be seen in FIG. 13. The program may cause the freckles to appear, via suitable progressive irradiation, either from the center of the zone towards the outside of the zone (FIGS. 13A to 13C), or from a sparse distribution to a dense distribution (FIGS. 14A to 14C), or from a distribution of small freckles to a distribution of large freckles, or in a random manner (not shown).

Irradiation may be sufficiently weak not to cause major development in the moment following its commencement.

In order to allow sufficiently slow development, the energy E of irradiation per second may be less than or equal to 0.5 E₀, preferably 0.2 E₀, where E₀ is the energy necessary per second to develop 80% of the thermally stable photochromic composition. It is possible for E≦0.2 E₀. It is considered that 80% of the thermally stable photochromic composition has been developed when the color change ΔE compared with the non-developed state corresponds to 80% of the maximum attainable color change.

Similarly, when it is to be erased, the energy E′ of irradiation per second may be 0.5 E′₀ or less, where E′₀ is the energy necessary per second to erase 80% of the thermally stable photochromic composition. It is possible for E′≦0.2 E′₀.

The system for processing light-sensitive makeup may be configured to analyze the color of the zone to be treated, then the result of this analysis may serve to automatically control the irradiation. As an example, the color may be analyzed after application of the thermally stable photochromic composition and before the desired appearance has been achieved. This may, for example, allow light-sensitive making up to be stopped automatically when the desired appearance has been achieved. The color may, for example, be measured by analyzing the color of the pixels of an image formed on the treated zone.

The system for processing light-sensitive makeup may be configured to carry out an analysis of pre-defined regions of the image and the irradiation may be controlled by adjusting the intensity of the irradiation in the various zones observed, as a function of the color in the corresponding regions. When the irradiation is carried out with an addressable matrix imager, then the irradiation in multiple pixels of the treated zone may be monitored precisely.

The irradiation may be constant or variable. In particular, it may be fairly strong for a given time, termed the “bring-up” time, then be weakened for a “fine tune” phase.

The system for processing light-sensitive makeup may be programmed to deliver the irradiation intermittently, an example being constant irradiation followed by a stop period, for example for a period of 30 seconds or less, and so on. The user may stop the process when satisfied.

When the user intervenes to stop the irradiation and then recommences it, the irradiation may be sufficiently slow to allow the user to see the color change, the irradiation changing, for example, at a rate of 3 or fewer units of E per second in CIE Lab space, for example approximately 2 units of E per second.

When the intensity of the irradiation is modulatable, the system for processing light-sensitive makeup may be provided with a control member to adjust the speed and/or the amplitude of the reduction or increase of the irradiation, for example a button, sensor, joystick, voice control interface, or control pad, which may act on the intensity of the irradiation, in particular upstream from the imager.

Depending on the implementation of the invention, the user may stop or throttle back the irradiation as desired in order to consider and/or observe the result.

Provision may be made for irradiation to be carried out as a function of the execution of an irradiation program by the system for processing light-sensitive makeup, and the user may either interrupt or pause the program during its execution, or change from one program to another. The program enabling the change in the irradiation over time to itself be defined or specified by the user, for example in order to adjust the rate of increase or reduction of the irradiation.

The increase or reduction of the irradiation intensity does not necessarily cause a change in the shape or extent of the image. Thus, in order to modulate the irradiation, it is possible to use electrical and/or optical systems that adjust the light flux produced, for example at least one filter, diaphragm, and/or polarizer, and/or a device for varying the electric power in order to control the source. The intensity of the irradiation may also depend on the gray level of the pixels of the image.

The system for processing light-sensitive makeup may be configured to automatically determine a progressive illumination program as a function of the light-sensitive makeup to be produced. As an example, if the light-sensitive makeup on a given zone consists in producing a color with fairly low saturation, the system for processing light-sensitive makeup may propose and/or apply a program specifying weak illumination. If the light-sensitive makeup on another zone consists in producing an intense color, the system for processing light-sensitive makeup may propose and/or apply a program consisting in illuminating more strongly at first then illuminating less strongly, to allow the user to fine tune the results of light-sensitive making up. The illumination carried out initially may be carried out with a flux that is at least double that which is subsequently applied.

In order to determine the intensity of the irradiation, the system for processing light-sensitive makeup may be based on a calculation of the dose to be applied in order to create the final light-sensitive makeup look and to apply a rule in order to deduce the illumination program therefrom. As an example, if it calculates that a dose of X J is required, it could rapidly apply 80% of X (over one second, for example) then apply the last 20% at 5% per second, for example.

As mentioned above, the system for processing light-sensitive makeup may be provided with an optical acquisition device that allows the color of the skin or other keratinous material to be measured either at the beginning of irradiation or during light-sensitive making up. It may use this information to compute or modulate the progressive illumination. As an example, it may use this information in order to identify the time when illumination needs to be reduced or stopped.

The sensor or sensors for the optical acquisition device may be monochrome or polychrome, with monopixel or multipixel measurement.

Information representative of the progress of light-sensitive making up may be transmitted to the user in various manners, for example by displaying a value representative of the color of the light-sensitive makeup being created, or a value representative of the degree of completion of the process, for example as a percentage. A color representing the measured color may also be displayed on the screen.

### Backtracking

The system for processing light-sensitive makeup may act to progressively reduce the intensity of the light-sensitive makeup, in order to cause one or more portions of the light-sensitive makeup to disappear or regress, by using illumination suitable for returning the photochromic agent or agents to a non-developed state.

In this manner, the user may retrace earlier steps and better adjust the final result. The system for processing light-sensitive makeup is advantageously produced so that the user may stop the backtracking when desired, restart light-sensitive making up, and so on.

For certain photochromic agents, for example selected from diarylethenes and fulgides, the light-sensitive makeup may be backtracked by replacing all or some of the UV illumination by visible illumination, for example white light.

The system for processing light-sensitive makeup is preferably configured so that this visible illumination extends over at least the same surface as the UV illumination.

A thermally Stable Photochromic Composition with Multiple Photochromic Agents

When the thermally stable photochromic composition comprises a plurality of photochromic agents with maximum sensitivities at respective different wavelengths, one or more of said photochromic agents may be selectively developed by adjusting the wavelength.

It is also possible to use a thermally stable photochromic composition with a plurality of different photochromic agents that are in the already-developed state and that are best erased at respective different wavelengths. Where appropriate, these photochromic agents may be developed by the same UV light, but have different erasure rates in the visible region that vary as a function of wavelength, such that by selecting wavelength, erasure of one photochromic agent rather than another is favored. Similarly, when the photochromic agents are capable of being developed by UV light, they may be developed at different rates depending on the wavelength in the UV region, and by adjusting this UV wavelength, development of one photochromic agent may be favored over others.

### Simulation of Change of Light-Sensitive Makeup

In one implementation of the invention, the system for processing light-sensitive makeup is provided with a system for simulating the change in the light-sensitive makeup look, in addition to or replacing a system for viewing the change in the light-sensitive makeup.

Thus, before and/or during light-sensitive making up, the user may observe this simulation and may use this to decide whether to slow down or stop the light-sensitive making up or even to backtrack.

The system for processing light-sensitive makeup may be configured to make it possible to simulate the result of light-sensitive making up after applying the thermally stable photochromic composition and before the desired appearance has been achieved. The progress of the simulation may be linked to the progress of the irradiation on the zone to be treated, whether it acts to develop the thermally stable photochromic composition or, on the contrary, to erase it. A simulation of the change in the appearance of the light-sensitive makeup may be displayed on a screen and/or projected onto the treated zone when the irradiator used makes that possible. The simulation may be projected in a light that does not cause the thermally stable photochromic composition to be developed or erased, at least over a short period, long enough for the observer to decide how to continue the treatment.

### Use of Tools

When the system for processing light-sensitive makeup includes an electronic imager, it may be controlled as a function of a tool manipulated by the user, the computer being able to modify the projected image and/or the intensity of the irradiation as a function of a movement of the tool. The tool may include a portion to be positioned in front of or on the zone to be treated or in front of or on a screen for viewing the zone to be treated. The tool may also control the movement of a pointer on a screen for viewing the zone to be treated or within the image formed on the zone to be treated.

The system for processing light-sensitive makeup may be configured to allow the user to control the particular zones that are to be treated with progressive irradiation, and to this end to make use of display means that may, for example, comprise a touch screen via which the user may control progress of the irradiation by pressing a particular region of the screen. More preferably, the touch screen is sensitive to the intensity of the pressure exerted by the user, and the system for processing light-sensitive makeup analyzes the pressure exerted on the screen and translates that pressure into the intensity of the light-sensitive makeup by controlling the intensity of the light and/or the duration of irradiation on the region corresponding to the zone to be treated. Thus, for example, a stronger pressure exerted on the touch screen is translated into an increased intensity of color.

In one implementation of the invention, the system for processing light-sensitive makeup may detect a tool placed on or in front of the touch screen, and the user may use the tool to adjust the light-sensitive makeup look.

As an example, the user may have several tools available, each provided with identification means, for example a bar code or an RFID chip, so that it is capable of being identified by the system for processing light-sensitive makeup. When the user takes a particular tool, it is recognized and each tool may be associated by the system for processing light-sensitive makeup with a particular type of makeup.

As an example, the user will have a plurality of tools corresponding to lines of makeup that are of varying thickness and/or varying intensities of color, or even different colors of makeup. The user takes the selected tool and may move it over the image on the display means in order to change the makeup look. A makeup simulation may appear on the viewing screen and after any required validation by the user, the makeup look appearing on the display means may be automatically created by light-sensitive making up performed by controlling the irradiator.

Adjustment and/or Modification of the Contents of the Image

In one implementation of the invention, a layer of light-sensitive makeup is developed with the same simulation image as that projected in visible light. To this end, the system for processing light-sensitive makeup may be configured to transmit an image to the zone to be treated, for example the face, representing the simulation, leaving it up to the user to lock this image on the face, or even to modify it. Then, once the image has been correctly adjusted and defined, the same optics or a parallel optical system is used to transmit an image that differs from the preceding image only in that it is formed with UV light or near UV light. In particular, the mask, negative, or matrix of addressable pixels used to define the image does not need to have been modified when changing the illuminant from visible light to UV light. To this end, a system for processing light-sensitive makeup provided with a UV source and a visible source, is suitable for use with one or two imagers.

If the image is obtained from a slide, it may be designed to allow an image to be produced both with the visible source and with an image with the UV source. To this end, the slide may be produced with a material that filters both UV and visible light.

If the image is obtained with at least one matrix of addressable pixels of an electronic imager, several configurations are possible:
two light sources, namely a UV source and a visible source, and a single matrix;
two light sources, respectively UV and visible, and two matrices, respectively UV and visible. The images are combined, for example with an X prism, or projected from sites located either side of the treated zone;
a light source emitting both in the UV and in the visible and a UV or visible imager with, for example, a dichroic mirror, a movable mirror, or a filter to select the outgoing radiation;
a light source emitting both in the UV and in the visible and two matrices, respectively for the UV and for the visible.

The projected image may be restricted to an outline or to a few reference points. The light-sensitive makeup then created may be inscribed within these points or this outline.

As an example, when treating the lips, two reference points may be employed. The user applies a layer of thermally stable photochromic composition. The two points are projected in a visible wavelength that is incapable of producing a light-sensitive makeup effect, for example at a wavelength in the range 450 nm to 800 nm, and preferably in the range 500 nm to 700 nm. The user positions these two points over the two corners of the lips. Once the positioning has been carried out, the UV irradiation forms an image of the lips included between these two points.

During UV illumination, the visible image may be cropped, reduced, or limited to the reference points or left in the same state as it was during the stage of projection in the visible.

Use of Light-Sensitive Makeup to Produce a Treatment Template on the Face then to Use it to Treat the Zone

A photochromic agent is applied to the zone, with the photochromic agent being selected, for example, from diarylethenes and fulgides, so as to have thermal stability that is sufficient for the image that is produced to hold for at least 20 seconds, and preferably for at least 1 minute. The template may be an image comprising points and/or reference lines, as shown in FIG. 11, which shows the lines defining the zones A, B, and C produced by light-sensitive making up. If necessary, an indicator is produced in the same way, for example an alphanumeric indicator, to inform the user about the composition and/or the applicator to be used in this zone.

The user may create the makeup look or other treatment as a function of the template that is thus developed. To this end, the user may use conventional makeup tools. The user may also use the template produced by the light-sensitive makeup to apply care products.

As an example, a diagnosis is carried out of zones requiring particular care, for example by image analysis and/or using one or more sensors that are sensitive to the condition of the skin, for example, and a map is produced of the treatment to be carried out, which map may be stored. Next, the system for processing light-sensitive makeup is used to cause a template to appear on the face where the zones requiring care are developed in color, at least temporarily. The user may then apply the care product or products to the zones that have been shown up.

Positioning of Reference Points on the Face, then Use of an Appliance Recognizing Said Points and Serving to Create the Makeup Look

The user may position reference points on the face either by using light-sensitive makeup, or by projecting the points and by marking them by hand by tracing the projection, e.g. by means of a pen.

Once the reference points have been placed on the zone to be treated, an appliance provided with means for reading the reference points may be used; it is preferably also capable of interpreting them if necessary. In one implementation, the appliance may be provided with a multipixel sensor, internal lighting, and applicator means, for example an inkjet printing head.

The points represent a line, for example. The user moves the appliance over the skin, which appliance is configured to analyze the surface optically. As an example, assuming that the zone to be treated is defined by a closed outline, when the appliance determines that it crosses a line a first time, it starts to deposit a colored material, then when it crosses a line a second time, it stops depositing the colored material.

When they are connected, the points may represent a shape. The appliance may be provided with means that may recognize these points and the corresponding shape. The appliance may deposit a colored material in order to produce this shape. To this end, the appliance may start from a model that it causes to coincide with the points by carrying out geometrical corrections.

The deposited colored material may define a curve or a surface inscribed within a curve.

The curvature of the line and/or the surface may be homogeneous, randomly heterogeneous, or geometrically heterogeneous, i.e. comprising a repeating pattern.

The points present on the zone to be treated may define a code. The appliance may be provided with means for interpreting the code corresponding to these points, and for applying a composition that is selected and/or applied in a manner that depends on the recognized code.

As an example, certain points are formed with a first pattern and others with a different pattern. The appliance applies two different compositions or the same composition in different concentrations as a function of the pattern that is detected.

### Photoprotective Composition

As disclosed above, a photoprotective composition may be applied to the thermally stable photochromic composition once the desired appearance has been achieved. This photoprotective composition may act as a screen to UV radiation when the irradiation that is used to develop the thermally stable photochromic composition is UV irradiation.

If necessary, the photoprotective composition may also act as a screen at least one predefined wavelength in the visible, with a view to limiting the risk of erasing a photochromic agent, where appropriate.

One or more optical agents may be used that provide the photochromic composition with a screening power F for solar radiation (290 nm to 400 nm) in the range 2 to 20, preferably in the range 4 to 10.

Where appropriate, the photoprotective composition may be a glossing composition, an oily or emollient composition, a mattifying composition, a cream blusher, a powder blusher, a polish, or a finishing composition.

### Optical Agents

### Optical Agents Forming a Screen to Radiation Serving for Development, in Particular UV or Near UV

The optical agent or agents mentioned above may be selected from screens and diffusing particles or other agents limiting the transmission of UV, especially UVA and/or UVB.

This or these optical agents may be selected from inorganic screens, in particular in particulate form and on a nanometric scale, and organic screens.

The optical agent or agents may be hydrophilic or lipophilic.

The organic filters may be selected from anthranilate derivatives, cinnamic derivatives, salicylic derivatives, camphor derivatives, benzimidazole derivatives, benzotriazole derivatives, benzalmalonate derivatives, imidazolines, bis-benzoazolyl derivatives, benzoxazole derivatives, triazine derivatives, benzophenone derivatives, dibenzoylmethane derivatives, beta, beta diphenylacrylate derivatives, p-aminobenzoic derivatives, polymer screens and silicone screens described in application WO 93/04665, dimers derived from alpha-alkylstyrene, 4,4-diarylbutadienes, and mixtures thereof.

The hydrophilic screens may be selected from those described in the application EP-A-0 678 292, for example 3-benzylidene 2-camphor, especially Mexoryl SX®.

Examples of lipophilic screens that may be mentioned are dibenzoylmethane derivatives, described in publications FR-A-2 326 405, FR-A-2 440 933, EP-A-0 114 607, Parsol® 1789 from Givaudan, Eusolex from Merck. It is also possible to mention 2-ethylhexyl 2-cyano-2,2-diphenylacrylate, known as octocrylene and available under the trade name Uvinul N 539 from BASF.

It is also possible to mention p-methylbenzylidene camphor, sold under the trade name Eusolex EX 6300 by Merck.

A screen selected from the following may also be used as the optical agent: benzophenone-3 (oxybenzone), benzophenone-4 (sulisobenzone), benzophenone-8 (dioxybenzone), bis-ethylhexyloxyphenol methoxyphenyl triazine (BEMT or Tinosorb S), diethylamino hydroxybenzoyl hexyl benzoate (Uvinul+), ethylhexyl methoxycinnamate, ethylhexyl salicylate, ethylhexyl triazone, methyl anthranilate (meradimate), (4-)methyl-benzylidene camphor (Parsol 5000), methylene bis-benzotriazolyl tetramethylbutylphenol (Tinosorb M), para-aminobenzoic acid (PABA), phenylbenzimidazole sulfonic acid (Ensulizole), polysilicone 15 (Parsol SLX), triethanolamine salicylate.

The optical agent used may also be formed by diffusing particles such as titanium or zinc oxide nanopigments that are suitable for use as a screen, with various surface treatments depending on the selected medium. The nanopigments have a typical mean dimension of 5 nm to 1000 nm.

The total concentration by weight of said optical agent(s) may lie in the range 0.001% to 30%, or even more with dry or near-dry formulas, relative to the weight of the photoprotective composition prior to application.

Preferably, screens or combinations are used in the composition that acts as a screen to radiation in the range from 320 nm to 400 nm, preferably in the range 320 nm to 420 nm.

### Optical Agents Intended to Limit the Propagation of Visible and Infrared Light Towards the Photochromic Agent or Agents

Although the optical agents acting as a screen to UV radiation serve to protect the non-developed zones, when using a thermally stable photochromic composition capable of being developed by UV irradiation, one or more optical agents that screen in the visible may also be applied to the thermally stable photochromic composition in order to protect the developed zones, and it may be advantageous to combine the two, namely screening in the UV and screening in the visible.

Many coloring agents or pigments may be used. In particular, it is preferred to use coloring agents with a color close to that of the skin, for example yellow coloring agents, orange coloring agents or mixtures that enable yellow, orange, ochre, brown, or chestnut hues to be produced or even red coloring agents that are preferably used either in small quantities or else mixed with a white or yellow diffusing agent, for example, to give the hue a pastel appearance such as pink or beige-pink. It is preferable to use coloring agent with a slightly pink hue for white skin, a slightly yellow hue for oriental skin, and chestnut or brown hues for skins that are termed black.

Alone or as a mixture, the coloring agents may have chromaticity close to that of the skin. They preferably have chroma C* (in the HVC* system) of less than 40.

The coloring agent or coloring agents may be selected from:
yellow pigments codified in the Color index with references CI 11680, 11710, 15985, 19140, 20040, 21100, 21108, 47000, 47005;
orange pigments codified in the Color Index with references CI 11725, 15510, 45370, 71105; and
red pigments codified in the Color Index with references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470.

In the photoprotective composition, pigment pastes of organic pigment may be used, such as the products sold by HOECHST with trade names:
JAUNE COSMENYL IOG: Pigment 5 t YELLOW 3 (CI 11710);
JAUNE COSMENYL G: Pigment YELLOW 1 (CI 11680);
ORANGE COSMENYL GR: Pigment ORANGE 43 (CI 71105).

Lakes may be used, in particular those known by the denominations D & C Red 21 (CI 45 380), D & C Orange 5 (CI 45370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Yellow 10 (CI 77 002).

The coloring agents may be ionic or neutral.

Natural coloring agents and pigments are particularly advantageous since they combine well with natural complexions and some of them lose their color over time. They are, for example, extracts from plants or natural molecules that have been artificially reproduced, being selected, for example, from melanine, anthocyans, polyphenols, porphyrins, and curcumin.

They may, for example, be pigments obtained by oxidation polymerization of indole and/or phenol derivatives, such as those described in the publication FR-2 679 771.

Coloring agents and pigments with an ionic nature that complements UV screens are particularly advantageous, for example coloring agents with an anionic function, such as certain food coloring agents and cationic filters.

It is also possible to use IR filters or compounds that, on reacting, become colored, for example DHA.

It is also possible to use a color-change colorant, for example a coloring agent of color that develops over time, if possible slowly, for example DHA or polyphenols, which tend to become progressively colored in contact with air. This enables the screening power of the photoprotective composition to develop progressively.

### Thermally Unstable Photochromic Agents

The optical agent used in the photoprotective composition may be a thermally unstable photochromic coloration agent. This does not serve to create the light-sensitive makeup look, but rather to protect it in the event of exposure to too intense a light, for example in very strong sunshine or an artificial effect such as the illumination used in television studios, certain medical treatments, certain cosmetic treatments such as tanning booths, for example, flash photography, or certain festive venues.

The thermally unstable photochromic agent takes on its color during very intense illumination and, in a certain manner, it may limit the visibility of the underlying light-sensitive makeup. However, since the thermally unstable photochromic agent rapidly regains its colorless form once the very intense illumination has been stopped, this phenomenon is transient.

Preferably, thermally unstable photochromic agents are used that lose at least half of their color in 60 seconds at 25° C. in darkness. In particular, inorganic thermally unstable photochromic agents are preferred.

Optical Agents that are Capable of Reflecting Incident Light

The optical agent used, in particular in order to attenuate UV or visible light, may be an optical agent forming a metal mirror or an optical agent based on a multilayer interfering structure or a diffraction grating.

The optical agents that are suitable for use alone or as a complement to the optical agents listed above are optical agents that are capable of reflecting incident light. The reflection occurs at the interface between the reflective layer and the propagation medium for the light wave. The material forming the reflective layer may have a refractive index of more than 1.5, if possible more than 1.8.

The optical agent may contain or be formed by a metal. As an example, a layer of silver is formed on applying the photoprotective composition by reducing a silver salt or by applying a dispersion of silver nanoparticles.

The degree of reflection of the photoprotective composition may be more than 5%, and if possible more than 10%. Preferably, it is less than 50%, in order not to vitiate the light-sensitive makeup results. As an example, the photoprotective composition may comprise a dispersion, which is either aqueous or ethanolic, of nanoparticles of silver, for example those from Nippon Paint that have a dimension of 10 nm to 60 nm depending on the sample and that are stabilized by a polymeric system. On drying, this stabilization does not prevent the particles from coming into contact and by means of these contacts of ensuring sufficient electrical conductivity to provide the final material with a reflective power close to that obtained with a silver mirror.

It is possible to use an optical agent comprising a multilayer interference structure.

This interference structure filters light by means of a phenomenon of destructive interference between the light waves reflected by the various layers of the structure.

The multilayer structure is preferably selected so as to have a high transmission factor in the visible, so that it does not produce a marked color in the visible and so that it has the desired transparency.

The multilayer structure may comprise alternating layers of low and high refractive indices. By way of example, the refractive index difference between the layers of high and low index is 0.1 or more, preferably 0.15 or more, more preferably 0.6 or more.

The number of layers in the above-mentioned multi-layer structure is preferably at least 2, more preferably 4 or 6, or even at least 12, which facilitates the production of a structure that is less sensitive to incident light and that presents the required selectivity. The multilayer structure may optionally be symmetrical and allow filtering of incident light irrespective of which is the principal face for light to enter the structure, as appropriate.

The material with a high refractive index may be mineral, for example titanium dioxide in the anatase or rutile form, an iron oxide, zirconium dioxide, zinc oxide, zinc sulfide, bismuth oxychloride, and mixtures thereof, or organic, being selected, for example, from: PEEK (polyetheretherketone), polyimide, PVN (poly(2-vinylnaphthalene)), PVK (poly(N-vinyl carbazole)), PF (phenolformaldehyde resin), PSU (polysulfone resin), PaMes (poly(alpha-methylstyrene)), PVDC, (poly(vinylidene chloride)), MeOS (poly(4-methoxystyrene)), PS (polystyrene), BPA, (bisphenol-A polycarbonate), PC (polycarbonate resin), PVB (poly(vinyl benzoate)), PET (poly(ethyleneterephthalate)), PDAP (poly(diallyl phthalate)), PPhMA (poly(phenylmethacrylate)), SAN (styrene/acrylonitrile copolymer), HDPE (high density polyethylene), PVC (poly(vinyl chloride)), NYLON®, POM (poly(oxymethylene) or polyformaldehyde), PMA (poly(methyl acrylate)), etc., and mixtures thereof.

The material with a low refractive index may be a mineral, for example selected from silicon dioxide, magnesium fluoride, aluminum oxide and mixtures thereof, or organic, for example selected from polymers such as polymethyl methacrylate or polystyrene, polyurethane and mixtures thereof.

In order produce the interference particles with a multilayer structure, the skilled person will in particular make reference to the many publications that deal with thin layer deposition, for example the article “Overcoated Microspheres for Specific Optical Powers” from the review Applied Optics, Vol. 41, no 6 dated Jan. 6, 2002, incorporated herein by reference, and to patents in the name of FLEXPRODUCTS.

The optical agent may comprise a diffracting structure, for example at least one diffraction grating, which may be a grating comprising a substantially repeating surface pattern so as to diffract light.

The period of the grating, and possibly its depth, determine the diffraction properties of the grating, inter alia. The mark space ratio of the diffraction grating may be selected to be unity.

Preferably, the period of the diffraction grating in at least one direction is advantageously sufficiently low to reduce the risk of creating colored effects in the photoprotective composition. The period of the grating is then advantageously selected so as not to diffract light in the visible region, especially in the range from 400 nm to 780 nm.

The maximum period of the grating serving to avoid diffraction orders in the visible may be determined at least approximately by the relationship:

$${n}_{1} sin θ + \frac{m λ}{Λ} = {n}_{2} sin Ψ ,$$

where θ is the angle of incidence measured relative to the normal to the plane of the grating, Ψ is the transmission angle, Λ is the period of the grating, m is diffraction order, and n₁ and n₂ are the refractive indices of the media in incidence and transmission respectively. n₁ and n₂ may be taken to be 1.5 to a first approximation. For θ=0°, the maximum period is λ/n₁=400/1.5 i.e. approximately 267 nm. Without limitation to the angle of incidence, the period is less than half. Thus, preferably, a period for the grating of 270 nm or less is selected, preferably 140 nm or less.

The depth d of the grating and its period A may be selected by successive tests in order to obtain a transmission minimum in the UVA, for example. Computation of the characteristics of the grating may be carried out vectorially, e.g. using the GSOLVER software from the GRATING SOLVER DEVELOPMENT COMPANY.

The layer or various layers used to produce the diffraction gratings may optionally be deposited on a substrate of an organic or inorganic nature, which substrate may be used as is or may then undergo a dissolution treatment.

The structure of the grating or gratings may thus be etched either into the bulk of a material, or else after depositing a material onto an organic or inorganic substrate that is spherical or lamellar in shape.

Etching may be carried out so that diffraction of the light in the visible region is minimized, in order to reduce color effects. The periodicity of the etching and its thickness determine the efficiency of the system in attenuating UV radiation.

The interference filter agent may optionally comprise two diffraction gratings extending in non-parallel directions, for example two substantially perpendicular directions, which gratings may in particular increase absorption in the UV of circularly polarized incident light and reduce the dependency of the screening performance of the filter angle of incidence.

The two diffraction gratings may have periods Λ₁ and Λ₂ that are substantially equal; in particular, both are 270 nm or less, preferably 140 nm or less.

The depths of the two diffraction gratings may also be substantially equal when they have surface relief, and that relief may create the periodic variation of the index of the grating.

The period of the grating may be constant or varying and the depth may be constant or varying. The grating may extend in a rectilinear or curvilinear direction.

The diffraction grating may comprise superimposed layers having different refractive indices. The diffraction grating may be produced at least partially from a dielectric material.

Various patterns may be used for the grating or gratings; they may, for example, have rectangular or triangular crenellations in section, or sinusoidal undulations, or stepped crenellations.

The diffracting structure may be formed over at least a portion of a principal face of the particle, preferably over the two principal faces of the particle.

The diffracting structure may comprise a protective and non-diffracting layer covering the grating or gratings.

Pigments having an interference effect and that are not fastened to a substrate may also be mentioned, such as liquid crystals (Helicones HC from Wacker), as well as interference holographic flakes (Geometric Pigments or Spectra f/x from Spectratrek).

The composition may comprise a mixture of interference elements for screening UVA and/or UVB, for example particles having diffraction gratings with different periods and/or depths.

### Optical Agents Capable of Transforming the Wavelength of Incident Light

The photoprotective composition may include a fluorescent compound.

The term “fluorescent” compound means a compound that absorbs light in the ultraviolet spectrum and possibly in the visible and that transforms the absorbed energy into fluorescent light with a longer wavelength emitted in the ultraviolet or visible part of the spectrum.

The compound may be an optical brightener that may be transparent and colorless, not absorbing visible light but only in the UV and transforming the absorbed energy into fluorescent light at a longer wavelength, for example 20 nm longer, or preferably 50 nm longer, or even 100 nm longer, that is emitted in the visible part of the spectrum; the color impression generated by said brighteners may thus be generated solely by predominantly blue purely fluorescent light with wavelengths of 400 nm to 500 nm.

Said compounds may be in solution or particulate.

The fluorescent compound may be a diketopyrrolopyrrole with formula:

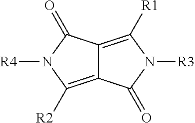

in which R₁, R₂, R₃ and R₄, independently of each other, represent a hydrogen atom; a halogen atom; a C₆-C₃₀ aryl group; a hydroxyl group; a cyano group; a nitro group; a sulfo group; an amino group; an acylamino group; a di (C₁-C₆)alkylamino group; a dihydroxy (C₁-C₆)alkylamino group; a (C₁-C₆)alkylhydroxy(C₁-C₆)alkylamino group; a (C₁-C₆) alkoxy group; a (C₁-C₆) alkoxy carbonyl group; a (C₁-C₆)carboxyalkoxy group; a piperidinosulfonyl group; a pyrrolidino group; an (C₁-C₆)alkylhalogeno (C₁-C₆)alkylamino group; a benzoyl(C₁-C₆)alkyl group; a vinyl group; a formyl group; a C₆-C₃₀ aryl radical that may be substituted with one or more groups selected from hydroxyl, linear, branched or cyclic C₁-C₆ alkoxy, linear, branched or cyclic alkyl containing 1 to 22 carbon atoms itself optionally being substituted with one or more hydroxyl, amino, C₁-C₆ alkoxy groups; a linear, branched or cyclic alkyl radical containing 1 to 22 carbon atoms, optionally substituted with one or more groups selected from hydroxyl, amino, linear, branched or cyclic C₁-C₆ alkoxy groups, optionally substituted aryl, carboxyl, sulfo groups, a halogen atom, said alkyl radical possibly being interrupted by a heteroatom.

The fluorescent compound may be a naphthalimide, with formula:

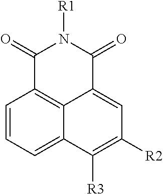

where

R₁, R₂, R₃, independently of each other, represent a hydrogen atom; a halogen atom; a C₆-C₃₀ aryl group; a hydroxyl group; a cyano group; a nitro group; a sulfo group; an amino group; an acylamino group; a di(C₁-C₆)alkylamino group, a dihydroxy(C₁-C₆)alkylamino group; a (C₁-C₆)alkylhydroxy(C₁-C₆)alkylamino group; a (C₁-C₆) alkoxy group; a (C₁-C₆)alkoxycarbonyl group; a C₁-C₆ carboxyalkoxy group; a piperidinosulfonyl group; a pyrrolidino group; a (C₁-C₆)alkylhalogeno(C₁-C₆)alkylamino group; a benzoyl (C₁-C₆)alkyl group; a vinyl group; a formyl group; a C₆-C₃₀ aryl radical optionally substituted with one or more groups selected from hydroxyl groups, linear, branched or cyclic C₁-C₆ alkoxy, linear, branched or cyclic alkyl containing 1 to 22 carbon atoms itself optionally being substituted with one or more hydroxyl, amino, C₁-C₆ alkoxy groups; a linear, branched or cyclic alkyl radical containing 1 to 22 carbon atoms, optionally substituted with one or more groups selected from hydroxyl, amino, linear, branched or cyclic C₁-C₆ alkoxy, optionally substituted aryl, carboxy, sulfo, a halogen atom, these 5 alkyl radicals possibly being interrupted by a heteroatom; the substituents R₁, R₂ and R₃ with the carbon atoms to which they are attached may form an aromatic or non-aromatic C₆-C₃₀ or heterocyclic cycle comprising a total of 5 to 30 links and 1 to 5 heteroatoms; said cycles may optionally be condensed, may optionally have a carbonyl group inserted, and being substituted or not substituted with one or more groups selected from C₁-C₄ alkyl groups, (C₁-C₄)alkoxy(C₁-C₄)alkyl, amino, di (C₁-C₄)alkylamino, halogen, phenyl, carboxy, and tri(C₁-C₄)alkylammonio(C₁-C₄)alkyl.

The fluorescent compound may be a stilbene derivative such as

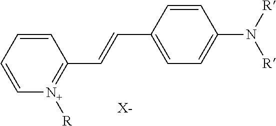

in which formula R represents a methyl or ethyl radical; R′ represents a methyl radical, X− represents a chloride, iodide, sulfate, methosulfate, acetate, perchlorate type anion.

An example of a compound of this type that may be mentioned is Photosensitizing Dye NK-557 sold by UBICHEM, where R represents an ethyl radical, R′ a methyl radical and X− an iodide.

The fluorescent compound may be a methyne derivative such as:

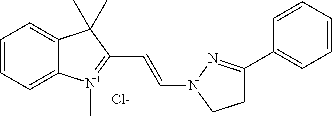

or
an oxazine or thiazine derivative with general formula:

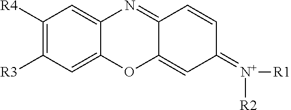

It is also possible to mention dicyanopyrazine derivatives (from Nippon Paint), naphtholactams, azalactone derivatives, rhodamines, and xanthenes.

It is also possible to use mineral (MgO, TiO₂, ZnO, Ca(OH)₂, etc) or organic (latex, etc) pigments or particles comprising said compounds at their core or on their surface.

The fluorescent compound may also be a semiconductor compound that has a fluorescent effect, for example in the form of small particles termed quantum dots.

Quantum dots are luminescent semiconductor nanoparticles that, under light excitation, are capable of emitting radiation at a wavelength in the range 400 nm to 700 nm. These nanoparticles may be fabricated in accordance with the methods described, for example, in U.S. Pat. No. 6,225,198 or U.S. Pat. No. 5,990,479, in the publications cited therein, and also in the following publications: Dabboussi B. O. et al “(CdSe)ZnS core-shell quantum dots: synthesis and characterization of a size series of highly luminescent nanocrystallites” Journal of Physical Chemistry B, vol 101, 1997, pp 9463-9475, and Peng, Xiaogang et al, “Epitaxial Growth of Highly Luminescent CdSe/CdS Core/shell Nanocrystals with Photostability and Electronic Accessibility” Journal of the American Chemical Society, vol 119, No 30, pp 7019-7029.

Preferred fluorescent compounds are those emitting orangey and yellow colors, for example.

Preferably, the fluorescent compound or compounds used as optical agents in the invention have a maximum reflectance in the wavelength range of 500 nm to 650 nm, preferably in the wavelength range from 550 nanometers to 620 nanometers.

Examples of fluorescent compounds are those belonging to the following families: naphthalimides; cationic or non-cationic coumarins; xantheno-diquinolizines (such as sulforhodamines in particular); azaxanthenes; naphtholactams; azlactones; oxazines; thiazines; dioxazines; azo, azomethinic or methinic type fluorescent polycationic coloring agents, used alone or as a mixture.

More particularly, the following may be mentioned:
Jaune Brilliant B6GL sold by SANDOZ and with the following structure:

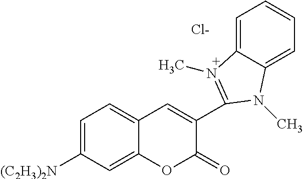

Basic Yellow 2, or Auramine 0 sold by PROLABO, ALDRICH or CARLO ERBA, with the following structure:

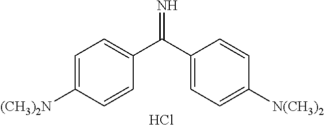

The fluorescent compounds used may be aminophenyl ethenyl aryl compounds, in which the aryl is a pyridinium, which may optionally be substituted, or another cationic group such as an imidizolinium, which may optionally be substituted.

As an example, a fluorescent compound may be used such as 2-[2-(4-dialkylamino)phenyl ethenyl]-1 alkylpyridinium, in which the alkyl radical of the pyridinium nucleus represents a methyl or ethyl radical; while that of the benzene ring represents a methyl radical.

An optical agent may contain several fluorescent groups on the same molecule. Examples are dimers such as:

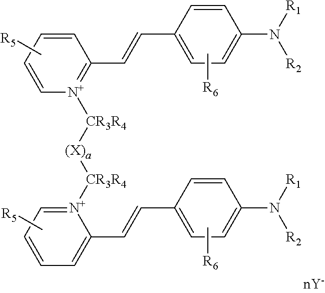

where R₁ and R₂, which may be identical or different, represent:
a hydrogen atom;
a linear or branched alkyl radical containing 1 to 10 carbon atoms, preferably 1 to 4 carbon atoms, optionally interrupted and/or substituted with at least one heteroatom and/or group comprising at least one heteroatom and/or substituted with at least one halogen atom;
an aryl or arylalkyl radical, the aryl group containing 6 carbon atoms and the alkyl radical containing 1 to 4 carbon atoms; the aryl radical optionally being substituted with one or more linear or branched alkyl radicals containing 1 to 4 carbon atoms, optionally interrupted and/or substituted with at least one heteroatom and/or group comprising at least one heteroatom and/or substituted with at least one halogen atom;
R₁ and R₂ may optionally be linked in order to form a heterocycle with the nitrogen atom and comprise one or more other heteroatoms, the heterocycle optionally being substituted with at least one linear or branched alkyl radical preferably containing 1 to 4 carbon atoms and optionally being interrupted and/or substituted with at least one heteroatom and/or group comprising at least one heteroatom and/or substituted with at least one halogen atom;
R₁ or R₂ may optionally be engaged in a heterocycle comprising the nitrogen atom and one of the carbon atoms of the phenyl group carrying said nitrogen atom;
R₂, R₄, which may optionally be identical, represent a hydrogen atom, or an alkyl radical containing 1 to 4 carbon atoms;
the R₅ moieties, which may optionally be identical, represent a hydrogen atom, a halogen atom, or a linear or branched alkyl radical containing 1 to 4 carbon atoms, optionally interrupted by at least one heteroatom; and
the R₆ moieties, which may optionally be identical, represent a hydrogen atom; a halogen atom; a linear or branched alkyl radical containing 1 to 4 carbon atoms, optionally substituted with and/or interrupted by at least one heteroatom and/or a group carrying at least one heteroatom and/or substituted with at least one halogen atom.

X represents:
a linear or branched alkyl radical containing 1 to 14 carbon atoms, or an alkenyl radical containing 2 to 14 carbon atoms, optionally interrupted by and/or substituted with at least one heteroatom and/or group comprising at least one heteroatom and/or substituted with at least one halogen atom;
a heterocyclic radical comprising 5 or 6 links, optionally substituted with at least one linear or branched alkyl radical containing 1 to 14 carbon atoms, optionally substituted with at least one heteroatom; with at least one linear or branched aminoalkyl radical containing 1 to 4 carbon atoms, optionally substituted with at least one heteroatom; or with at least one halogen atom;
an aromatic or diaromatic radical, which may optionally be condensed, separated or not separated by an alkyl radical containing 1 to 4 carbon atoms, the aryl radical or radicals optionally being substituted with at least one halogen atom or with at least one alkyl radical containing 1 to 10 carbon atoms optionally substituted with and/or interrupted by at least one heteroatom and/or group carrying at least one heteroatom;
a dicarbonyl radical;
the group X possibly carrying one or more cationic charges; and
a being equal to 0 or 1.

The Y− moieties, which may optionally be identical, represent an organic or mineral anion, with n being a whole number equal to at least 2 and at most the number of cationic charges present in the fluorescent compound.

Other dimers are possible, such as those in which the attachment point is formed between the two non-cationic groups or, for example those in which the pyridinium group is replaced by another arylcationic group such as an imidazolinium group.

The dicyanopyrazine family may also supply compounds that fluoresce in the orange and are of interest to the invention.

Pigments that fluoresce in the orange may also be used. An example is the Sunbrite-SG2515 yellow orange pigment sold by SunChemical.

### Application of the Photoprotective Composition

The user may apply the photoprotective composition to the whole of the treated zone, spreading the layer of thermally stable photochromic composition generously or, on the contrary, in a localized manner over only certain zones, as illustrated in FIG. 12. In this figure, the light-sensitive makeup has been created with a thermally stable photochromic composition PC completely covered by the photoprotective composition PP. The photoprotective composition may, for example, be localized to the edges of the zone coated with the thermally stable photochromic composition, thereby surrounding the light-sensitive makeup patterns when the light-sensitive makeup is less extensive than the layer of thermally stable photochromic composition.

The photoprotective composition layer may also take the form of a flexible film to be bonded to the keratinous material, for example the skin. The substance of the film may act as an optical agent and/or the film may contain at least one optical agent dispersed in the substance of the film. The film may also carry a coating containing the optical agent, for example in the form of an impression or a multilayer interference structure.

The user may apply said film over the whole of the thermally stable photochromic composition layer, or could cut the film to cover only the non-developed zones, not covering the developed zones.

An automatic cutting system may be used that, starting from the content of the light-sensitive makeup, e.g. its contour, cuts the protective film to the suitable shape. The user then places the cut protective film over the non-developed zones.

In another implementation of the invention, the photoprotective composition is deposited by transfer, by applying a support sheet carrying at least one optical agent to the zone to be treated. The user brings the sheet into contact with the keratinous material coated with the thermally stable photochromic composition and then uses friction or other means such as heat or a solvent to cause the optical agent or agents to be transferred onto the thermally stable photochromic composition layer.

In one implementation of the invention, the photoprotective composition layer is reversible, i.e. it is possible for the user to remove it without removing the first layer of thermally stable photochromic composition.

To this end, the first layer may be formulated so that it is water-resistant or resistant to a mixture of water and surfactant, and the second layer may be formulated so that it is not water-resistant or resistant to a mixture of water and surfactant.

It is also possible to produce a peelable second layer. To this end, a second layer may be used, forming a cohesive coating before or after application to the first layer. The second layer, when it is peelable, comprises an elastomeric material, for example.

In one implementation of the invention, the second layer is less adherent to the first layer, for example by the thermally stable photochromic composition making use of low surface tension compounds such as silicone or fluorinated compounds. In another implementation of the invention, an intermediate non-stick layer is interposed between the first and the second layer, facilitating removal of the photoprotective composition layer.

Particularly when it is reversible, the photoprotective composition layer may have a very high screening power F, for example 20 or more.

A single layer containing the optical agent or agents or several layers containing several different optical agents may be deposited.

As an example, a single layer may be deposited that ensures that the thermally stable photochromic layer is protected from UV and visible light.

It is also possible to deposit a specific UV protective layer and an additional layer for additional protection in the UV and/or in the visible, said additional layer comprising a coloring agent or a thermally unstable photochromic agent, for example.

It is also possible to use one UV protective layer and an additional layer comprising a fluorescent compound ensuring additional protection in the UV.

In a particular possibility, there is applied a multilayer film, comprising a first layer of thermally stable photochromic composition and a second layer that is photoprotective comprising an optical agent forming a screen against the development radiation for the thermally stable photochromic composition. This film may be self-supporting or it may be applied by transfer.

The thermally stable photochromic composition may be applied as is to the keratinous material, or it may be on a base layer, in particular a base layer as defined below.

The second composition may be applied directly to the thermally stable photochromic composition layer or to an intermediate layer between the two, as mentioned above. The second composition may itself be coated with an additional layer where appropriate.

### Choice of Ingredients for the Various Layers

In one implementation of the invention, two successively applied layers, for example the layer of thermally stable photochromic composition and the layer of photoprotective composition, or the base layer and the layer of thermally stable photochromic composition, or the layer of thermally stable photochromic and the layer intended to form a material protecting the light-sensitive makeup, may be physically complementary, allowing or facilitating the second layer to grip onto the first and/or allowing or facilitating spreading of the second layer on the first.

It may be advantageous for there to be ionic natures that are complementary. Thus, for example, the first layer may contain an anionic polymer and the second then contains a cationic compound, for example a cationic filter, a cationic coloring agent or a cationic fluorescent compound. The opposite is also possible.

It may also be advantageous for the surface tensions to be complementary. Thus, the first layer may have a first surface tension of preferably more than 40 mN·m⁻¹ [millinewton per meter], for example by using at least one hydrophilic polymer. The second layer may have a lower second surface tension than the first, preferably less than 40, for example by using a mainly oily, silicone, or fluorinated composition, or by using an aqueous composition into which one or more surfactants has been introduced.

The ingredients (solvents, adhesives, etc) for the second layer may be selected so that they are not solvents of the first.

As an example, an organic solvent (ethanol, acetone, alkyl acetate, carbonaceous oils (for example isododecane), volatile silicones (for example D5), etc) may be selected for the first layer and an aqueous or hydroalcoholic solvent may be selected for the second layer, or vice versa.

It is also possible to select two organic solvents or two aqueous solvents for the two layers, provided that, on drying the first layer, a transformation takes place. As an example, a first layer containing a latex is used. On drying, the latex coalesces and renders the first layer inert to application of the second layer. It is also possible to use a first layer containing a low hydrosolubility acrylic/acrylate copolymer rendered hydrosoluble by neutralization with a volatile base such as ammonia. After drying the first layer, the ammonia will evaporate and render the first layer water-resistant.

### Base Layer

A base layer of a photoprotective first composition may be applied to the keratinous material, the base layer containing at least one optical agent that is capable, at least temporarily, of forming a screen at a wavelength λ, especially a wavelength within the range 320 nm to 440 nm, and said base layer may have a thermally stable photochromic second composition applied thereto that is capable of being developed by exposure at least to radiation of wavelength λ; the optical agent or agents may be selected from those indicated above.

By way of example, and at least while it is being applied, the photoprotective composition applied as a base layer has a screening power F against solar radiation of at least 2, preferably 5 or 10.

Using the base layer may reduce the risk of staining the skin by rendering migration of the photochromic agent or agents of the thermally stable photochromic composition towards the subjacent keratinous material more difficult.

This migration may be further slowed or even prevented when the first and second compositions are not miscible with each other, one of the compositions being aqueous, for example, and the other non aqueous, or vice versa, in order to form two phases.

Thus, it is possible to select the ingredients (solvents, adhesives, etc) for the second composition that are not solvents for the thermally stable photochromic composition and vice versa. As an example, an organic solvent is selected from alcohols or ketones, for example, in particular ethanol or acetone, alkyl acetate, carbonaceous oils, in particular isododecane, or volatile silicones for the photoprotective second composition and an aqueous or hydroalcoholic solvent for the thermally stable photochromic first composition, or vice versa.

It is also possible to select two organic solvents or two aqueous solvents for the two compositions, such that a transformation takes place on drying. As an example, a first composition containing a latex may be used. On drying, this composition coalesces and renders the layer inert to application of the thermally stable photochromic composition.

The base layer may be formed on a surface that is more extensive than the thermally stable photochromic composition. This facilitates application of the thermally stable photochromic composition, since the user no longer needs to worry about making the outlines of the two applied compositions correspond precisely.

When the thermally stable photochromic composition is suitable for being developed by exposure to UV radiation, then the optical agent contained in the base layer is preferably a non-photostable sunscreen, with a photostability index of 80% or less.

One resulting advantage is that when a base layer is applied to the skin, the user is not completely prevented from tanning, even if the extent of the base layer goes substantially beyond that of the thermally stable photochromic composition. During exposure to the sun, the base layer may then lose its capacity to screen UV, which enables the user to tan progressively at least in the zone not covered by the thermally stable photochromic composition. If the sunscreen were photostable, users might fear applying the base layer too extensively, for fear of leaving trace marks in their tan.

In order to measure the photostability of a sunscreen, it is diluted in a C₁₂-C₁₅ alkyl benzoate solvent with trade name FINSOLV®. The screen Parsol 1789 is an example of a non-photostable screen, having a photostability of the order of 30%, defined as being the ratio between the screening power after one hour's exposure to UVA radiation produced by an irradiator from SUNTEST divided by the initial screening power.

The base layer may be applied well before the light-sensitive makeup, for example more than 15 minutes before, which may have the effect of leaving the base layer time to dry and to render it insoluble or nearly insoluble in the layer applied over it, as mentioned above. Further, on drying, the base layer may optionally form a relatively smooth surface, facilitating application of a layer of thermally stable photochromic composition with uniform thickness. The skin is thus smoothed, and so the second layer may be thinner and there is a reduced risk of non-uniform thickness, which might give rise to unattractive visual effects following development.

If necessary, at least one intermediate layer may be applied to the base layer in order to place it between the thermally stable photochromic composition and the base layer.

This intermediate layer may have the effect of improving the hold of the thermally stable photochromic composition on the base layer or, on the contrary, of facilitating removal during makeup removal, for example. The intermediate layer may be a layer of a polymer or wax.

In particular, the intermediate layer need not function as a screen at the wavelength λ for developing the thermally stable photochromic composition.

Further, a layer of another composition may be applied beneath the base layer to facilitate its adhesion to the skin. Thus, the base layer need not be directly in contact with the skin. In a variation, the base layer is applied directly to the skin or other keratinous material.

### Mechanical Protection of Light-Sensitive Makeup

At least one layer of thermally stable photochromic composition may be applied to the keratinous material, and by means of a second composition or by means of added energy, it may form a material that mechanically protects the light-sensitive makeup in the layer of thermally stable photochromic composition.

It is also possible to deposit at least one covering layer on the layer of thermally stable photochromic composition that enables a material ensuring mechanical protection of the light-sensitive makeup to be formed.

The light-sensitive makeup look may be created before or after forming the material providing the light-sensitive makeup with mechanical protection, by selectively developing the layer of thermally stable photochromic composition.

Improving the mechanical hold of the light-sensitive makeup may delay degradation of the image formed and the loss of sharpness of the image over time is slowed. Further, the light-sensitive makeup is rendered less sensitive to rubbing and to movements. The risk of transferring thermally stable photochromic composition onto clothing or other regions of the body is also reduced.

Thus, a more durable light-sensitive makeup may be created on zones such as zones covered with clothing, for example the back, stomach, breasts, legs, or buttocks.

Said material may be formed by solvent evaporation, or by a polymerization or cross-linking reaction, which does not necessarily need to be complete. Surface hardening, by polymerization and/or cross-linking, may prove sufficient to improve the hold.

The material providing mechanical protection of the light-sensitive makeup is advantageously transparent.

When the material covers the layer of thermally stable photochromic composition, the material forms a wear layer that, by wearing bit by bit during the day, protects the light-sensitive makeup.

When it covers the thermally stable photochromic composition layer, the material may also contribute to the esthetics of the light-sensitive makeup, by providing an additional optical effect, for example a magnifying or coloration effect.

When the thermally stable photochromic composition offers the possibility of erasing the light-sensitive makeup by irradiating the layer of thermally stable photochromic composition at a wavelength that differs from that used to develop the thermally stable photochromic composition, the material may improve its hold without in any way preventing the light-sensitive makeup from being removed if that is desired; the user does not need to remove the makeup completely for this purpose.

In order to form the material that provides the light-sensitive makeup with mechanical protection, it is possible to use polymerizable and/or cross-linkable compounds in the thermally stable photochromic composition and/or in the covering layer.

The thermally stable photochromic composition may contain all of the polymerizable and/or cross-linkable compounds serving to form the material. Optionally, the irradiation used to develop the thermally stable photochromic composition serves for polymerization and/or cross-linking.

The thermally stable photochromic composition may also contain a first agent that may potentially polymerize and/or cross-link. After or before development of the photochromic agents of the thermally stable photochromic composition, a second compound is applied that, by association with the first, may carry out the polymerization or cross-linking. The irradiation may also possibly serve to bring about polymerization and/or cross-linking.

In other implementations, the second composition is applied after applying the thermally stable photochromic composition and creating the light-sensitive makeup look.

The covering layer may be applied either before or after irradiation serving to develop the photochromic agent or agents. Its mean thickness may be at least 2 μm [micrometer], if possible at least 5 μm if the material is rather hard or elastomeric, preferably at least 10 μm if the material has a rather soft modulus of elasticity.

When the keratinous material is covered by a single layer that comprises the light-sensitive makeup, the thickness of said layer is preferably more than 5 μm, more preferably 10 μm. The thickness is preferably less than 1 mm.

When the thermally stable photochromic composition incorporates all or some of the compounds that may potentially cross-link, it is possible in a second stage, before or after drying the first composition, to apply a second compound that causes cross-linking or is necessary for cross-linking. The thickness of the second layer (expressed after evaporation of any solvents) is preferably equal to at least 20% of the thickness of the first layer, preferably more than 50% of the thickness of the first layer. The thickness of the second layer is preferably more than 5 μm.

When the thermally stable photochromic composition does not include any potentially cross-linkable compounds, it is possible in a second stage to apply a second composition containing the compounds that produce cross-linking. The thickness of the second layer (expressed after evaporating off any solvents) is preferably equal to at least 10% of the thickness of the first layer, and preferably more than 30% of the thickness of the first layer. The thickness of the second layer is at least more than 5 μm, preferably less than 1 mm.

Polymerization and/or cross-linking allowing formation of the material may be chemical or physical.

Chemical Polymerization and/or Cross-Linking

The term “chemical cross-linking” means that a compound, whether alone, or by reaction with a second compound, or by the action of radiation, or by the supply of energy, is capable of creating covalent chemical bonds between the molecules. The result is an increase in the cohesion of the material including said compound.

The compound may be a simple molecule, or it may already be the result of a combination of several molecules, for example oligomers or polymers. The compound may carry one or more reactive functions.

Preferred molecules are those that, after cross-linking, provide a solid and/or deformable but elastomeric material.

The chemical functions may react with another function of the same nature or that may react with another chemical function.

Reaction with Another Function of the Same Nature

These are, for example, ethylenic functions, in particular acrylates, acrylics, methacrylates, methacrylics, or styrene.

In order to react, these molecules generally require an external form of activation, for example light, heat, the use of a catalyst, or a combination with photoinitiators and possibly photosensitizers intended to broaden the spectrum of action of the photoinitiators. Photopolymerizable and/or photo-cross-linkable compositions are described, for example, in patents CA-A-1 306 954 and U.S. Pat. No. 5,456,905.

It is possible to use polymeric compounds carrying ethylenic functions as described in patent EP-A-1 247 515.

The ethylenic functions may be activated by an electron-attracting group in order to accelerate the reactions and render the supply of any external activation redundant. This is typical of the ethylcyanoacrylate monomer, for which the sole presence of a catalyst such as water allows the reaction to occur.

The ethylenic functions may be moderately activated, for example by an electron-attracting group. The advantage is that the reaction requires an external activation, which is important when controlling the initiation and the yield of the reaction, but does not require a photoinitiator. For example, it may be a cyanoacrylate monomer, in particular a cyanoacrylate monomer in which the group carried by the ester function contains at least 2, if possible 4 carbonaceous concatenations.

Molecules requiring external activation such as light but not requiring a photoinitiator are preferred. Thus, molecules that are capable of reacting by photodimerization, such as those described in the patent EP-A-1 572 139, are particularly preferred, in particular those carrying functions such as:

1) stilbazoliums:

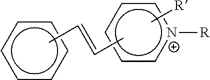

where
R represents a hydrogen atom, an alkyl or hydroxyalkyl group; and
R′ represents a hydrogen atom or an alkyl group;

2) styrylazoliums:

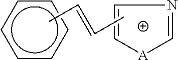

where

A designates a sulfur atom, an oxygen atom, or a NR′ or C(R′)₂, group, R and R′ being as defined above;

3) chalcone;

4) (thio)cinnamate and (thio)cinnamamide;

5) maleimide;

6) (thio)coumarin;

7) thymine;

8) uracil;

9) butadiene;

10) anthracene;

11) pyridone;

12) pyrrolizinone;

13) acridizinium salts;

14) furanone;

15) phenylbenzoxazole;

16) styrylpyrazine.

The reactions carried out on another function of the same nature are not limited to reactions involving ethylenic functions.

Compounds that may react by condensation are also preferred, such as:
siloxane groups, in particular dialkoxy- or dihydroxy-silane functions, trialkoxy- or trihydroxysilane functions. It is possible to use molecules carrying alkyltrialkoxysilane or dialkyltrialkoxysilane functions, in particular alkylalkoxysilane functions where the alkyl group carries a hydrosolubilizing function such as an amine, for example a molecule such as aminotriethoxysilane or aminotriethoxysilane, or molecules carrying such functions. In addition to small molecules based on siloxanes (monomers or oligomers), compounds with a larger mass may be used, in particular those described in patent FR-A-2 910 315;
sol-gels based on titanium.

With these molecules, it is possible to control initiation and reaction yield.

Compounds capable of reacting by oxidation are also preferred, such as aromatic compounds carrying at least two hydroxyl functions, or a hydroxyl function and an amine function, or a hydroxyl function, for example cathecol or dihydroxyindole. The oxidizing agent may be oxygen from the air or another oxidizing agent such as hydrogen peroxide, for example.

Reaction with Another Function

The molecules that react in such circumstances have two types of functions that are complementary. They may be systems in which molecules carrying functions FA are brought into contact with molecules carrying functions FB that are capable of reacting with the functions FA.

They may also be molecules that carry one or more functions FA and one or more functions FB on the same structure.

The function FA may be selected from the following, for example:
epoxide;
aziridine;
vinyl and activated vinyl, in particular acrylonitrile, acrylic and methacrylic esters;
crotonic acid and esters, cinnamic acid and esters, styrene and derivatives, butadiene;
vinyl ethers, vinylketone, maleic esters, vinylsulfones, maleimides;
carboxylic acid anhydride, hydrochloride and esters;
aldehydes;
acetals, hemi-acetals;
aminals, hemi-aminals;
ketones, alpha-hydroxyketones, alpha-haloketones;
lactones, thiolactones;
isocyanate;
thiocyanate;
imines;
imides, in particular succinimide, glutimide;
N-hydroxysuccinimide esters;
imidates;
thiosulfate;
oxazine and oxazoline;
oxazinium and oxazolinium;
C₁ to C₃₀ alkyl or C₆ to C₃₀ aryl or aralkyl halides with formula RX, with X═I, Br, Cl;
unsaturated, carbonaceous or heterocyclic ring halides, in particular chlorotriazines;
chloropyrimidine, chloroquinoxaline, chlorobenzotriazole;
sulfonyl halide: RSO₂—Cl or —F, R being a C₁ to C₃₀ alkyl.

By way of illustration, the following molecules carrying functions with a group FA may be mentioned:
methylvinyl ether and maleic anhydride copolymer, in particular sold by ISP with the trade name Gantrez, for example;
glycidyl polymethacrylate, in particular sold by Polysciences;
glycidyl polydimethylsiloxane, in particular sold by Shinetsu (reference X-2Z-173 FX or DX);
epoxy polyamidoamine, for example sold by Hercules with the trade name Delsette 101, Kymene 450 from Hercules;
epoxy-dextran; and
polyaldehyde polysaccharides obtained by oxidation of polysaccharides using NaIO₄ (Bioconjugate Techniques; Hermanson G T, Academic Press, 1996).

The function FB may be selected from XHn functions with X═O, N, S, COO and n=1 or 2, especially alcohols, amines, thiols and carboxylic acids.

Examples of molecules that carry FB type functions are as follows:
PAMAM dendrimer, in particular sold by Dendritech, D S M, Sigma-Aldrich (STARBURST, PAMAM DENDRIMER, G(2, O) from DENDRITECH);
dendrimer with hydroxyl functions, in particular sold by Perstorp, D S M, (example: HBP TMP core 2 Generation PERSTORP);
PEI (polyethylene-imine), in particular sold by BASF, with the trade name Lupasol;
PEI-Thiol;
polylysine, in particular sold by Chisso;
HP cellulose, such as KLUCELEF from AQUALON);
amino-dextran, for example sold by Carbomer;
amino-cellulose, for example those described in WO-01/25283 from BASF;
PVA (polyvinylacetal), for example AIRVOL 540 from AIRPRODUCTS CHEMICAL;
amino PVA, for example sold by Carbomer; and
chitosan.

This second case also includes molecules that may react by hydrosilylation:

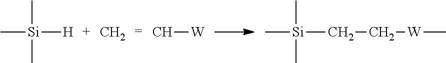

(W represents a carbonaceous or silicon-containing chain, for example).

Details of the two ingredients, the commercially available molecules, the conditions for the catalysts and the conditions of use are described in patent application FR-A-2 910 315.

In one particular possibility, a molecule that is already present on the skin or excreted by the skin is used as a reagent or catalytic agent. It is typically water, which may assist in the cyanoacrylate reaction, for example, or in certain reactions involving siloxanes.

In another particular possibility, a molecule is used as the reagent or catalytic agent that is present in ambient air. It is typically oxygen that is involved in the cross-linking reaction of certain oils such as siccative oils, in particular siccative vegetable oils such as linseed oil, China wood oil (or tung oil), oiticica oil, vernonia oil, poppy-seed oil, pomegranate oil, calendula oil or alkyd resins. The reactions may be accelerated by using catalysts such as cobalt, manganese, calcium, zirconium, zinc, strontium, lead, lithium, iron, cerium, barium, or tin salts in the form of the octoate, linoleate or octanoate, for example.

In anther particular possibility, molecules are used that bind with each other by rearranging. Thus, it is possible to use molecules that carry an internal disulfide. By opening the internal disulfide and reacting said disulfides, it is possible to create new covalent bonds between the molecules.

Catalysts may be used to accelerate the reactions. As an example, metal salts such as manganese, copper, iron, platinum, titanates or enzymes such as oxidases or laccases may be used.

With chemical functions that react with another function of the same or a different nature, several modes of application are possible.

As an example, all of the ingredients that react are incorporated in the thermally stable photochromic composition, or all of the ingredients are incorporated in the thermally stable photochromic composition with the exception of one or more compounds, for example one of the compounds, or a catalyst. It may be that none of the ingredients are incorporated in the thermally stable photochromic composition; they are all applied at once or at different times, after application of the thermally stable photochromic composition and preferably after creating the light-sensitive makeup look.

### Physical Cross-Linking

Cross-linking may be physical when ingredients are used that are capable of creating durable physical bonds between the molecules and endowing the final material with water resistance. These bonds, which are non-covalent, are of the ionic or hydrogen type.

Examples that may be mentioned are mixtures with a di- or poly-valent type salt, for example a calcium, zinc, strontium, or aluminum salt.

As an example, a compound A such as an alginate derivative and a compound B such as a calcium salt may be mixed. By way of example, the alginate derivative is contained in the thermally stable photochromic composition. In a second stage, an aqueous solution of calcium chloride is applied in the form of a spray, for example, in order to cause cross-linking.

Molecules that are capable of creating strong hydrogen bonds may also be mentioned, such as polysiloxane and polyurea block copolymers, and in particular those with formulae:

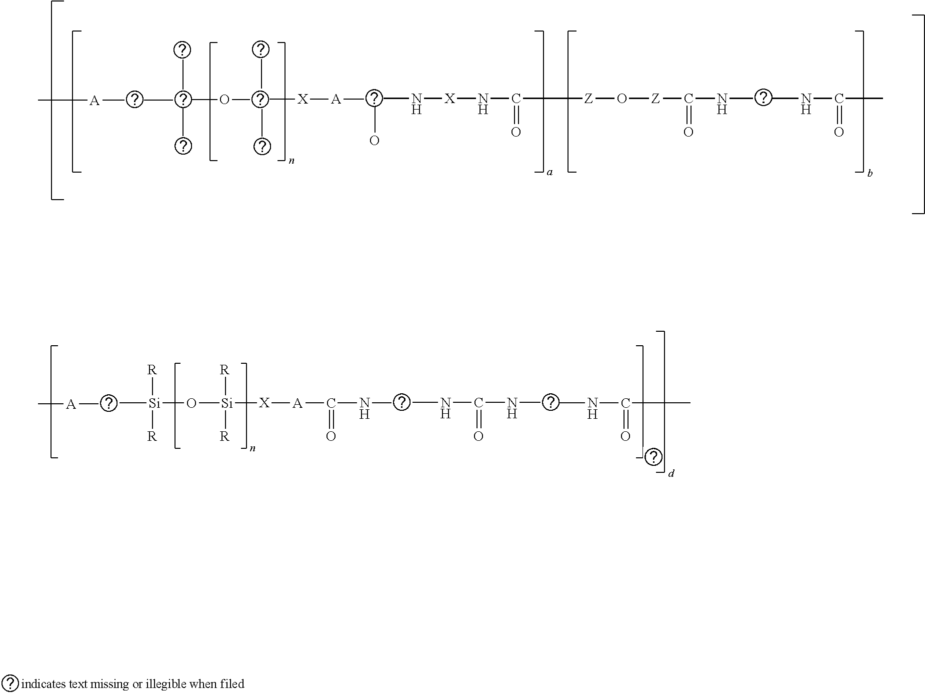

where:
R represents a monovalent hydrocarbon radical containing 1 to 20 carbon atoms, which may be substituted with one or more fluorine or chlorine atoms;
X represents an alkylene radical containing 1 to 20 carbon atoms, in which the non-neighboring methylene units may be replaced by —O— radicals;
A represents an oxygen atom or an amino radical —NR′—;
Z represents an oxygen atom or an amino radical —NR′—;
R′ represents hydrogen or an alkyl radical containing 1 to 10 carbon atoms;
Y represents a bivalent hydrocarbon radical, if necessary substituted with fluorine or chlorine, containing 1 to 20 carbon atoms;
D represents an alkylene radical, if necessary substituted with fluorine, chlorine, C₁-C₆ alkyl, or C₁-C₆ alkyl ester, containing 1 to 700 carbon atoms, in which the non neighboring methylene units may be replaced by the radicals —O—, —COO—, —OCO— or —OCOO—;
n is a number from 1 to 4000;
a is a number that is at least 1;
b is a number from 0 to 40;
c is a number from 0 to 30; and
d is a number greater than 0.

Details of the functions, commercially available molecules, and the implementation conditions are given in patent EP-A-0 759 812.

### Cross-Linking Compounds Leading to the Formation of a Particularly Resistant Coating

Irrespective of whether the cross-linking is chemical or physical, the cross-linking compounds may be selected so that they provide the best possible resistance, in particular to water and moisture.

Thus, it is possible to produce highly hydrophobic coatings, in particular to treat the parts of the body that perspire the most, such as the bust or the armpits, for example.

As an example, a first reactive ingredient FA of the polyol type may be used, such as a cellulose derivative, and a second reactive ingredient FB of the perfluoroalkyltriethoxysilane type. Under such circumstances, the application is carried out in two stages. The polyol is introduced into the thermally stable photochromic composition. A coating composition containing the ingredient FB is applied to the thermally stable photochromic composition.

In another example, a system is employed that is capable of producing a cross-linked coating; it also contains hydrophobic particles. An illustration of these combinations is the combination of hydrophobic particles with condensation techniques or hydrosilylation techniques such as those described in patent FR-A-2 910 315. The solid particles that may be used may be of mineral or organic origin, porous or non porous, colored or not colored. They may have any morphology, preferably spherical. The particles may be naturally hydrophobic, which is the case with PTFE powder, for example, or they may be rendered hydrophobic by coatings, in particular of hydrocarbons, silicones, fluoro compounds or fluorosilicones.

It is also possible to produce coatings that provide better resistance to sebum and to fats, based on oxide or zinc salts, for example, or coatings that are rendered more resistant to elongation or to tearing. These improvements may be of use in applications to parts of the body that move the most, such as the lips, the hands, the armpits, the neck, or any zones close to joints.

The elongation strength may be acquired by using cross-linking ingredients that, for example, produce a material with an elastomeric nature. It is also possible to integrate non reactive compounds into the composition or compositions, providing an elastomeric nature, for example an elastomeric polymer such as a deproteined natural latex or fibers.

One particular possibility is to impregnate a woven or nonwoven fabric with cross-linking ingredients. A woven or nonwoven fabric may be applied to the skin before or during or after application of the light-sensitive makeup composition. Impregnating the composition into the fabric provides mechanical strength.

It is also possible to combine the fabric and the light-sensitive makeup composition then, once produced, to apply it to the skin with or without using an adhesive.

Lubricating active ingredients may be incorporated in the compositions, in particular solid lubricants such as boron nitride or aluminum, for example.

It is also possible to integrate solid fillers, in particular fillers that are hydrophilic or rendered hydrophilic, such as metal oxide particles, metal hydroxide particles, metal carbonate particles, or organic particles. These fillers may provide additional abrasion resistance.

### Covering Layer Forming Wear Layer

The coating layer may form a mechanical protective material above the thermally stable photochromic composition layer and act as a wear layer.

The coating layer is then advantageously cohesive, after evaporating off any solvents, and it may be applied before or after irradiation.

The term “cohesive” means that the layer is resistant on contact. As an example, if a flat probe with a surface area of 1 cm² [square centimeter] is brought towards the coating layer, so that it comes into contact with a pressure of 10 N/cm² [newton per square centimeter], then the probe is withdrawn after a contact time of 5 seconds, it must not entrain matter. Thus, oily compounds are excluded.

The coating layer is not sticky once the solvents have been evaporated off. The term “not sticky” means that the layer offers no resistance to withdrawal. As an example, if a flat probe with a surface area of 1 cm² is brought towards the layer so that it comes into contact with a pressure of 10 N/cm², and then it is withdrawn after a contact time of 5 seconds, it must not require a resistive force to achieve that withdrawal. Thus, compounds known as PSA (pressure sensitive adhesive) are excluded.

The material forming the coating layer may have a modulus of elasticity of less than 500 MPa [megapascal] and more than 100 kPa [kilopascal], preferably in the range 200 MPa to 1 MPa.

Its mean thickness is at least 1 μm, if possible at least 2 μm if the material has a modulus of elasticity of more than 10 MPa. Its mean thickness is at least 2 μm, if possible at least 5 μm if the material has a modulus of elasticity of less than 10 MPa.

When the material forming the coating layer is elastomeric, i.e. has a maximum deformation of at least 400% before rupture and has an elastic recovery of at least 90% after waiting for 1 minute, the mean thickness is preferably at least 1 μm, even if the modulus of elasticity is less than 10 MPa.

The term “elastic recovery” means the degree of return to the initial length of a specimen after 40% tensile deformation then release of the load. Thus, if the initial length of the specimen is L0, and the length after 40% tensile deformation and release of the load is L(t), the recovery R(t) at time t from the release is equal to:

100×(1−(L(t)−L0)/L0)/0.4).

Thus, if L(t)=L0, then R(t)=100.

If L(t)=1.4×L0, then R(t)=0

The recovery test is carried out by initially preparing a specimen approximately 200 μm thick, 6 cm in length, and 1 cm wide. If necessary, the specimen is optionally produced on a support film; its mechanical impact is judged to be small compared with the mechanical properties of the specimen.

The specimen is subjected to a tensile deformation of 40% of its length at a rate of 0.1 mm/s [millimeter per second]. Next, the load is released and 1 minute is allowed to pass.

Preferably, the coating layer is applied with a solvent that is very different from that used for the thermally stable photochromic composition layer. However, this condition may be circumvented, in particular when using cross-linking or coalescent compounds for the thermally stable photochromic composition layer.

As an example, if the thermally stable photochromic composition layer contains a latex with a glass transition temperature, Tg of <40° C. and water, for example, the coating layer may also be water-based.

If the thermally stable photochromic composition layer contains a solvent and a compound that is capable of cross-linking, such as those described above, for example, the coating layer may contain the same solvent.

In order to assist in producing the coating layer properly, prior to its application, light or heat may be supplied, for example. It is also possible to deposit an intermediate layer produced, for example, from a resin or any other product that aids adhesion, such as an adhesive or certain powders, in particular those that assist the upper layer in gripping because of their grain size.

After application of the coating layer, light or heat may be supplied.

The coating layer may be eliminated progressively. Thus, the light-sensitive makeup layer is not altered over time and precision of the light-sensitive makeup is fully retained.

### Ingredients in the Coating Layer Forming the Wear Layer

The compounds that may be used in forming said coating layer are polymers, for example poly(meth-)acrylics, poly(meth-)acrylates, polyurethanes, polyesters, polystyrenes or copolymers in the soluble or dispersed form, for example selected from Mexomer, ultrahold Strong DR 25, 28-29-30, Gantrez, Amerhold DR 25, amphomer, Luviset Si Pur, AQ 38, or AQ 48.

The polymers may carry side or terminal groups in order to adjust their hardness. As an example, the material forming the coating layer may comprise acrylate polymers with silicone functions, such as VS 80, for example.

The polymers may be natural polymers or modified natural polymers, for example polyosic polymers, such as guar gums, carouba gums, or cellulose derivatives, such as HPMCP [hydroxypropylmethylcellulose phthalate] or proteins.

The polymers may be hydrocarbon polymers.

The polymers may be silicones such as silicone gums, for example.

Since the intrinsic qualities of the majority of polymers cannot always provide the required hardness, it may be useful to add a plasticizer.

In addition to the normally used plasticizers, for example glycol ether (tripropylene glycol monomethyl ether (known as PPG3 methyl ether, from DOW CHEMICAL) or glycerin, certain non-volatile solvents may be included, such as propylene carbonate, alcohols, silicone or carbonaceous oils.

The quantities of plasticizers are calculated as a function of the polymer and its intrinsic qualities. Typical values are as follows (% relative to the weight of polymer):

| | Glycol ether | Glycerin |
|---|---|---|
| Ultrahold Strong DR 25 (BASF) | 5% | 10% |
| Mexomer (Chimex) | 4% | 8% |
| AQ 48 (Eastman Chemicals) | 1% | 2% |
| Luviset Si Pur (BASF) | 3% | 5% |
| VS 80 (3M) | 5% | 10% |

Mineral or organic particles may be included in the composition, which may prolong the service life of the wear layer without, however, causing it to lose its qualities. The particles do not cause tightness in the skin but may, however, cause the phenomena of flaking or balling. Thus, preferably a concentration by weight of 40% of particles is not exceeded (particles capable of coalescing not included).

Rheological agents that aid application may be included.

It is also possible to include spreading agents such as surfactants or certain solvents with a boiling point in the range 80° C. to 200° C. These solvents have the advantage of slowing down caking of the composition while being eliminated over time.

### Concentrations and Thicknesses After Drying

The concentrations of the various ingredients may be adjusted so that the thicknesses after drying are, taking into account the quantities applied, in agreement with the specifications given above.

As an example, assuming that 20 mg/cm² of fluid composition for spreading is applied, and that the composition contains 10% dry matter, then it is possible to deposit approximately 2 mg/cm². If the density is approximately 1, this corresponds to a thickness of approximately 20 μm.

In another example, if it assumed that spraying of an aerosol composition comprising 20% dry matter is carried out 30 cm from the face for 4 seconds, then approximately 0.4 g [grams] be deposited over 400 cm², i.e. 1 mg per cm². If the relative density is approximately 1, then the thickness of the deposited layer will be approximately 10 μm.

Thus, depending on the modes of application and the galenical forms, the dry matter concentrations may be from 1% to 50%.

The coating composition may be dry.

### Other Ingredients

In addition to the ingredients mentioned above, each composition may contain ingredients that make the following possible or easier: distributing over the keratinous material, more particularly the skin; providing skin care, comfort, for example odor or softness; aiding in elimination on washing, for example one or more surfactants; limiting penetration of the ingredients into the skin, for example astringents; or supplying other cosmetic functions, for example moisturizing, color, shine, and/or limiting the impact of ultraviolet screening, for example a self-tanning agent or a vitamin D activator.

### Makeup Removal

When removing makeup, the user may leave traces of the non-developed thermally stable photochromic composition. However, these traces may be caused to be developed subsequently, for example after a few hours have passed in ambient light. At that time, it may be difficult for the user to start removing the makeup again.

In order to overcome this problem, it may be advantageous during or after makeup removal to apply at least one optical agent that forms a screen at least one wavelength λ and that acts to develop the thermally stable photochromic composition.

Said optical agent may be re-applied several times, where appropriate.

The optical agent may be part of a makeup removal composition.

The term “forming a screen at the wavelength λ” means that the optical agent attenuates radiation with a wavelength λ by a factor of at least 2, the measurement being carried out using apparatus that may measure the absorption spectrum by restricting the irradiating light to a zone with a wavelength centered around wavelength λ, as detailed above. Applying the optical agent during, and preferably after, makeup removal prevents traces of photochromic agent from being developed and reduces the risk of staining the keratinous material or clothing.

The keratinous material should not be washed during the hour following application of the optical agent. Later, when the user washes, the non-developed traces of photochromic agent protected by the optical agent can be eliminated.

Another advantage linked to application of the optical agent is that, when a fresh light-sensitive makeup look is created, it prevents certain non-developed parts of the preceding light-sensitive makeup that are still present from being developed during exposure to the radiation used to create the fresh light-sensitive makeup look.

The optical agent may be applied after removing the makeup. The optical agent may also form part of the formulation of a makeup removal composition used for makeup removal.

The wavelength λ may fall within the UV or near UV spectrum (290 nm to 400 nm), in particular in the range from 320 nm to 440 nm.

The makeup removal composition may be a conventional makeup removal product based on surfactants or a particular makeup removal product adapted to compounds from the thermally stable photochromic composition, and may include a solvent, for example ethyl or butyl acetate, acetone, ethanol or mixtures thereof, and more generally any solvent selected from cosmetically acceptable organic solvents (acceptable tolerance, toxicology, and feel). These organic solvents may represent 0% to 98% of the total composition weight. They may be selected from the group constituted by hydrophilic organic solvents, lipophilic organic solvents, amphiphilic solvents, and mixtures thereof. Examples of hydrophilic organic solvents that may be mentioned are linear or branched lower mono alcohols containing 1 to 8 carbon atoms such as ethanol, propanol, butanol, isopropanol, or isobutanol; polyethylene glycols containing 6 to 80 ethylene oxide moieties; polyols such as propylene glycol, isoprene glycol, butylene glycol, glycerol, or sorbitol; mono- or di-alkyl isosorbides of alkyl groups that contain 1 to 5 carbon atoms; glycol ethers such as diethylene glycol mono-methyl or mono-ethyl ether, and propylene glycol ethers such as dipropylene glycol methyl ether.

Examples of amphiphilic organic solvents that may be mentioned are polyols such as derivatives of polypropylene glycol (PPG), such as the esters of polypropylene glycol and fatty acid, or PPG and fatty acid such as PPG-23 oleyl ether or PPG-36 oleate. Examples of lipophilic organic solvents that may be mentioned are fatty esters such as diisopropyl adipate, dioctyl adipate, alkyl benzoates, isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, di-(2-ethylhexyl) succinate, diisostearyl malate, 2-octyldodecyl lactate, glycerin triisostearate, or diglycerin triisostearate.

The makeup removal composition may also comprise:
an oil, for example in the form of a microemulsion;
a pH agent if the compound or compounds used to maintain the photochromic agent or agents on the skin are pH-sensitive, as is carbopol, for example; or
an ionic liquid.

Examples of anionic surfactants that may be used alone or as a mixture in the makeup removal composition that may in particular be mentioned are alkaline salts, ammonium salts, amine salts or amino alcohol salts of the following compounds: alkoylsulfates, alkoylether sulfates, alkoylamide sulfates and ether sulfates, alkoylarylpolyethersulfates, monoglyceride sulfates, alkoylsulfonates, alkoylamide sulfonates, alkoylarylsulfonates, α-olefin sulfonates, paraffin sulfonates, alkoylsulfosuccinates, alkoylethersulfosuccinates, alkoylamide sulfosuccinates, alkoylsulfosuccinamates, alkoylsulfoacetates, alkoylpolyglycerol carboxylates, alkoylphosphates/alkoyletherphosphates, acylsarcosinates, alkoylpolypeptidates, alkoylamidopolypeptidates, acylisethionates and alkoyllaurates.

The alkoyl or acyl radical in all of these compounds generally designates a chain containing 12 to 18 carbon atoms.

It is also possible to mention soaps and fatty acid salts such as oleic, ricinoleic, palmitic, stearic acids, coprah oil acids or hydrogenated coprah oil acids and in particular salts of amines such as amine stearates; acyl lactylates of acyl radical that contains 8-20 carbon atoms; and carboxylic acids of polyglycolic ethers with formula:

Alk-(OCH₂—CH₂)ₙ—OCH₂—COOH

in the acid or salt form, in which the substituent Alk corresponds to a straight chain containing 12 to 18 carbon atoms and in which n is a whole number in the range 5 to 15.

Examples of non-ionic surfactants that may be used alone or as a mixture and that may be mentioned in particular are: alcohols, alkoylphenols and polyethoxylated, polypropoxylated or polyglycerolated fatty acids with a fatty chain containing 8 to 18 carbon atoms; copolymers of ethylene oxide and propylene oxide, condensates of ethylene oxide and propylene oxide on fatty alcohols, polyethoxylated fatty amides, polyethoxylated fatty amines, ethanolamides, esters of fatty acids with glycol, esters of fatty acids with sorbitan, which may optionally be oxyethylenated, esters of fatty acids with saccharose, esters of fatty acids with polyethylene glycol, phosphoric triesters, esters of fatty acids with glucose derivatives; alkylpolyglycosides and alkylamides of aminated sugars; the condensation products of an α-diol, a monoalcohol, an alkoylphenol, an amide or a diglycolamide with glycidol, or a precursor of glycidol.

The makeup removal composition that contains the optical agent may be formulated so as to allow the optical agent to be deposited on rinsing, for example by coacervation effect, this effect being capable of being obtained, for example, by using surfactants and polymers with complementary ionic natures, for example PC/PA, TC/TA, TC/PA, TA/PC, possibly with amphoteric and non-ionic surfactants to facilitate deposition. PCs are typically compounds such as cationic guar gums (Jaguar C13S, for example) or artificial compounds such as JR 400 or ionene. TCs are typically quaternary chain compounds (in particular trimethylammonium groups) and fatty chain compounds (C₆ to C_{H}). PAs may be multianionic polymers such as acrylate or methacrylate polymers or copolymers or polymers containing sulfonic groups. TAs are anionic surfactants such as carboxylic or sulfate or sulfonic surfactants (LES, LS).

The makeup removal composition may be applied using any suitable support, in particular one that is capable of absorbing, for example a fibrous makeup removal disk, for example woven or nonwoven, felt, cotton-wool, flocked film, sponge, or towelette; the support used for makeup removal is advantageously eliminated after the makeup removal operation.

The makeup removal composition may be contained in a receptacle and withdrawn each time the makeup is to be removed. In a variation, the makeup removal composition impregnates the support used for makeup removal, the support then possibly being packaged, for example in sealed packaging. After using the makeup removal composition, the keratinous material does not need to be rinsed. In a variation, it may be rinsed. Rinsing may be carried out using running water, for example, without adding soap.

### Projecting an Image and Creating a Makeup Look by Tracing the Projection

When the cosmetic treatment that is performed as a function of the projected image, in accordance with the invention, is making up, the projection of the image may be created by any irradiator, but preferably an electronic addressable matrix imager as defined above.

Since the making-up treatment may take a certain amount of time, the imager may be associated with a tracker system of tracking the face, so as to correct the projected image, e.g. the axis of the projection, in real time. To do this, the tracker system may observe one or more reference points, e.g. by means of an optical acquisition device.

In particular when projection is for simulating a makeup look, it may be in real color or in false color. A false color may help the user to see the projection clearly. For example, the color of the projection may be green, white, blue, or bright red, since these are colors that contrast sharply with the colors of the face. An especially bright projection could be used to help the user to see the image projected on the face clearly.

Preferably, the projection takes place while the user is in the dark or in a dimly-lit room, so as to make the projected image stand out better.

The projection system used is preferably provided with means that make it possible to adjust the intensity of the projection. Where appropriate, adjustment may be automatic by using an optical acquisition device such as a sensor that analyzes ambient light and that adjusts the intensity of the projected light so as to exceed that of ambient light.

The projection system used is preferably provided with means that make it possible to select the projected image as described below for the system for processing light-sensitive makeup. In one implementation of the invention, a tailored image is used starting from the individual intended to receive the makeup, or from a model such as a celebrity or an individual of given style, the images possibly being derived from made-up or non made-up people. It is also possible to use images derived from drawings, painting, sketches, or caricatures to generate the projected image.

The system used may be configured to take an image, for example using the optical acquisition device, optionally to extract a portion corresponding to the zone to be treated, and where appropriate to rectify this image to thereby improve the result once projected.

The system used is preferably configured to allow the user, starting from an image projected on the face, to rectify the shape, for example by enlarging, or shrinking in one or two dimensions. The modifications may also be more complex. Thus, for example, it is possible to rectify a portion of the image, stretch a particular zone, change the size of the lines, etc. For this, it is possible to use the tools normally present in software for producing and editing images, such as Photoshop® for example. Where appropriate, the image may be edited by feedback via the optical acquisition device.

The projection system used is preferably configured to make it possible to focus the projected image on the face of the individual.

The projection system used enables the image to be changed, either in the form of a succession of images, or else in the form of an animated image that changes progressively. A changing projection enables the individual who applies the makeup to proceed with a succession of strokes. Where appropriate, the projected image may change on the basis of an analysis of the image of the zone during treatment. For example, the system used may determine that a region has just been treated entirely, and may thus display information enabling a different region to be treated, or the same region to be treated with a different applicator and/or composition. The system may deliver information in visual or audible manner, explaining to the user which colors and makeup tools should be used at each stage in applying light-sensitive makeup.

The makeup tools are either those normally used, or they are adapted tools. For example, they are tools that are moved into contact with the zone being treated, so as to deposit a cosmetic composition thereon, in particular makeup. Preferably, the tools are made in such a manner as to be transparent or translucent, at least in part, so as not to create too much shadow on the zone being treated. The tool may be an applicator member such as a flocked or foam endpiece, e.g. carried by an optionally-opaque fine stem 201, as shown in FIG. 17, and preferably with a shape that does not deform the optical path, e.g. a section that is flat. Where appropriate, application is performed using an applicator member 200, itself non-opaque, e.g. made of a transparent or translucent material. In the figure, the user deposits composition with the applicator 202 tracing a line T projected by the imager.

The treatment performed during projection of the image may also be remote application of a cosmetic composition or a care produce, in particular makeup, by spraying.

The composition that is deposited by contact with the zone being treated or that is sprayed from a distance may also be a composition that causes a reaction in a layer that has been applied beforehand. Thus, the image may be projected by the system onto a zone that is coated with a composition that is capable of changing in appearance when put into contact with a developer agent, the composition including a colored indicator that is sensitive to folding or that is sensitive to a metallic salt. The developer agent is applied selectively by the user to the keratinous material as a function of the projected image, e.g. tracing the outlines defined.

The treatment performed during projection of the image may be a selective application of energy for developing a composition and causing it to change in appearance, as described above. For example, the zone to be treated is coated with a thermally stable photochromic composition, and a light-sensitive makeup look is created by subjecting the composition to appropriate illumination in different locations, e.g. UV using a system for processing light-sensitive makeup as described above. In a variant, irradiation may also come from a portable user-manipulated irradiator distinct from the irradiator used to project the image.

### EXAMPLES

### Formula I (Photochromic Composition)

diarylethene* (sold with reference DAE-TZ by Yamada Chemical (Japan)): 0.4%
PMMA (polymethyl methacrylate sold by Wako Pure Chemical Industries, Ltd (Japan)): 10%
acetone: qs 100%

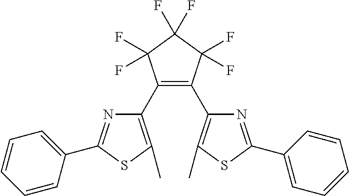

### Negative

A negative may be created for the light-sensitive makeup as follows. A PowerPoint® file is produced representing a grid in which each square has a side of approximately 3 mm. This file is then printed onto a transparency using a laser printer. Where it is not printed, the transparency allows UV to pass through in the UVA activation band, at about 365 nm.

### Irradiator

An irradiator such as that shown in FIG. 6 is produced, integrating: the lamp emitting in the UV, said lamp emitting white light; the green filter; the adjustable collimation optic; and the movable mirror 304.

### Support

The support used is a white polyurethane material representing the texture of the skin, sold by Beaulax under the trade name Bioskin ref #white 061031-2.

### Tests

The tests were carried out in ambient light in a closed room.

### Example 1

The photochromic composition was applied on the support, then allowed to dry for 1 minute. Projection took place 20 cm from the support. The first switch was used to activate projection of the simulation in visible light. The imaged projected on the surface was observed. Focus and positioning were adjusted, then the second switch for emitting in UV light was actuated to activate the light-sensitive makeup and form the design of the negative with the photochromic composition.

### Example 2

The same irradiator as above was used, except for the negative and the fact that the green filter was removed from the apparatus.

The negative was formed on a transparent medium by using a filter composition (Parsol 1789) and an image in the form of a stencil representing the lips in negative, the surface of the lips not being covered by the filter.

Then, four red dots were drawn on the transparent medium with a felt tip pen, representing the two corners of the lips and the top and bottom points of the lips.

The photochromic composition was applied on a support simulating the lips, then the irradiator was placed 20 cm from the support.

The first switch was used to activate projection of the simulation. The image projected on the surface comprised four red dots. Focus and positioning were adjusted so that the red dots merged with the shape of the lips. Then, the second switch was actuated to activate the light-sensitive makeup and form a lip design with the photochromic composition.

### Example 3

The same irradiator was used as in example 1.

The negative was made as follows.

A photograph was taken of a person having a few red or pink marks on the face, and was printed on paper. A transparent medium was placed on top and the entire surface of the medium was blackened except for the marks.

In this example, the photochromic composition was a blue diarylethene and a yellow diarylethene such as those indicated above.

The photochromic composition was applied on the surface where the marks were situated, then the irradiator was placed 20 cm from the surface.

The first switch was used to activate projection of the simulation. The image projected on the surface was formed by bright dots. Focus and positioning were adjusted so that the bright dots merged with the real marks. Then, the second switch was actuated to activate the light-sensitive makeup.

A green color was developed exactly on the marks, thereby limiting their visibility.

### Example 4

The same test was performed as in example 2, except that the image on the transparent medium represented only the outline of the lips.

Using the irradiator, a light-sensitive makeup was created that represented the outline of the lips in red. The inside of the outline was then made-up with a lipstick.

The advantage of this method is that it enables people who have difficulty in achieving precise makeup to obtain a makeup look that is satisfactory.

### Example 5

The same test as in example 2 was performed, except that the photochromic composition used was based on a blue diarylethene.

The resulting light-sensitive makeup represented only the outline of the lips, in the form of a blue dotted line. That pattern was used to create the makeup look.

### Example 6

A Logitech Quick Cam Pro 9000 camera and a UV matrix projector are connected to a portable computer.

A semi-transparent 60/40 mirror is positioned so that light emitted by the projector goes through the mirror and incident light coming parallel to the projector is reflected at 90° toward the camera.

The UV matrix projector comprises five parts:
a DLP component configured to operate with UV light, such as a DMD 0.7 inches XGA from Texas Instruments,
a 10 Watt UV lamp emitting in the range 340 nm to 400 nm,
a controller to control mirrors of the DLP, such as a DDC 4100 Digital controller from Texas Instruments,
an interface and power supply for the above components, such as Texas Instruments DAD 2000 DMP Power and Reset Driver,
an optical system to focus light from the matrix projector at about 30 cm.

The portable computer is programmed to capture a camera image, process said image to detect the face, determine zones to be treated, such as the forehead and the two cheeks, then detect within each zone the darkest areas. The captured image is displayed on the screen and the darkest areas are highlighted, when they exist. If the software does not detect dark zones, a message such as “no treatment is possible” is displayed.

The software is configured to prompt the user to accept or decline to treat said zones. If the user accepts, the software controls the DLP to illuminate, in a selective way, the light areas located between the dark areas. Then, the image displayed by the DLP is projected with UV light.

A control panel allows the user to stop the illumination, to lower the intensity or to restart the illumination.

Before operating, the user sets the optical system to optically align the camera and the UV projector and to check the magnification so that the fields are superposed.

Photochrome Product (Kept in Dark):

| | |
|---|---|
| Diarylethene magenta sold under reference DAE-TZ by the | 0.06 g |
| company YAMADA CHEMICALS | |
| Diarylethene yellow sold under reference DAE | 0.28 g |
| 2BT by the company YAMADA CHEMICALS | |
| Diarylethene blue sold under reference DAE | 0.06 g |
| 2BT by company YAMADA CHEMICALS | |
| PMMA (methyl methacrylate polymer) sold by the | 10 g |
| Company WAKO PURE CHEMICALS Ltd | |
| Acetone | 89.6 g |

Protective Product:

Sun DNA Guard SP50 by LANCOME.

The method is performed on a person having skin imperfections such as beige dyschromia.

The photochrome product is applied over the one or various areas having imperfections, over the whole area without restricting to the imperfections, for example 0.5 g for an area such as the forehead. The person to be treated is positioned along the optical axis of the UV projector and the camera (positioned 90° offset relative to the projector).

The image acquisition is started after the person has closed her eyes.

An interface showing “proposed areas” allows selection of the one or more areas to be treated. The illumination then starts. The operator monitors the progress of the make-up and reduces, when desirable, the illumination intensity using the control panel, when the color starts to become homogeneous, i.e. the areas with the skin imperfections tend to obtain a color similar or the same as the remaining areas, then the operator stops the illumination, using the control panel. If the result is satisfying, the operator applies the protective product over all areas where the photochrome product has been applied.

### Example 7

One uses the same device as in example 6.

The computer is programmed to:
capture an image from the camera,
process the image to delimit the face and detect the forehead and the two cheeks,
detect in the these areas the lightest zones,
display on the screen the image from the camera and highlight the lightest zones, if any.

If the program does not detect such zones, a corresponding message is displayed.

The software is configured to prompt the user to accept or decline to treat said zones. If the user accepts, the software controls the DLP to illuminate, in a selective way, the light areas located between the dark areas. Then, the image displayed by the DLP is projected with UV light.

A control panel allows the user to stop the illumination, to lower the intensity or to restart the illumination.

Before operating, the user sets the optical system to optically align the camera and the UV projector and to check the magnification so that the fields are superposed.

A subject having depigmentation (vitiligo) is treated.

The same photochrome product as in Example 1 is applied on the one or more areas having imperfections by targeting the imperfection but extending beyond the borders around the imperfections by about 2 cm, for example in an amount about 0.1 g/cm².

The person to be treated is positioned along the optical axis of the UV projector and the camera (positioned 90° offset relative to the projector).

The image acquisition is started after the person has closed her eyes.

An interface showing “proposed areas” allows selection of the one or more areas to be treated. The illumination then starts. The operator monitors the progress of the make-up and reduces, when desirable, the illumination intensity using the control panel, when the color starts to become homogeneous, i.e. the areas with the skin imperfections tend to obtain a color similar or the same as the remaining areas, then the operator stops the illumination, using the control panel. If the result is satisfying, the operator applies the protective product over all areas where the photochrome product has been applied.

### Example 8

The same device as in Example 6 is used.

The computer is configured to capture an image of the camera and detect the outline of the face, the lips, the eyes and eyebrows.

It generates:
a mask corresponding to the lips, the mask being an image whose outline is the natural outline of the lips, and/or
a mask corresponding to the area between the eye and the eyebrow, the mask being an image whose outline is bordered at the bottom end by the top of the upper eyelid and at the top end by the bottom of the eyebrows.

The image of the mask is projected with visible light. The operator can check the quality of the proposed image.

The computer is equipped with an image touch-up software, which allows refining each of the masks, for example to redraw the outline of the image.

The program is configured to enable the user to select a fill pattern for the images, and the fill pattern of the images can be uniform or contain a geometric pattern.

An interface allows the user to accept or decline the treatment of one or more areas.

Before operating, the user sets the optical system to optically align the camera and the UV projector and to check the magnification so that the fields are superposed.

The photochrome product is applied on the lips, in an amount for example about 0.5 g.

The photochrome product is applied over the area between the eyes and the eyebrows. The upper eyelids are covered with the product.

Blue photochrome product (kept in dark):

| | |
|---|---|
| Diarylethene blue sold under reference DAE | 0.3 g |
| 2BT by company YAMADA CHEMICALS | |
| PMMA (methyl methacrylate polymer) sold by the | 10 g |
| Company WAKO PURE CHEMICALS Ltd | |
| Acetone | 89.7 g |

Red photochrome product (kept in dark)

| | |
|---|---|
| Diarylethene magenta sold under reference DAE-TZ | 0.06 g |
| by the company YAMADA CHEMICALS | |
| Diarylethene yellow sold under reference DAE | 0.28 g |
| 2BT by the company YAMADA CHEMICALS | |

Protective product:

| Sun DNA Guard SP50 by LANCOME. | |
|---|---|
| Acetone | 99.66 |

The person to be treated is positioned along the optical axis of the UV projector and the camera (positioned 90° offset relative to the projector).

The red or blue photochrome product is applied on the lips (for example about 0.5 g) and in the area between the eyes and the eyebrows. The upper eyelids are also covered with the product.

The image acquisition is started after the person has closed her eyes.

An interface showing “proposed areas” allows selection of the one or more areas to be treated. If necessary, an interface allow the user to touch up the image.

The illumination then starts. The operator monitors the progress of the make-up and reduces, when desirable, the illumination intensity using the control panel. When the color starts to reach to desired color, then the operator stops the illumination, using the control panel. If the result is satisfying, the operator applies the protective product over all areas where the photochrome product has been applied.

### Example 9

The device is the same as for example 6 and the programs are the same as for example 8.

In addition, the computer is equipped with a program that generates a mask starting from a decorative image that will be positioned taking into account the elements of the face.

For example, the eyes are detected and a mask is created with either a line following the outline of the eyes or a pattern positioned in alignment with the eyes. In the latter case, the program detects the position of each eye, identifies the main axis of the eyes and positions, at the external corner of the eye, a pattern adjacent to the corner of the eyes, along the main axis.

The projector is controlled to project the mask and make the decorative images appear.

The UV matrix projector comprises five parts:
a DLP component configured to operate with UV light, such as a DMD 0.7 inches XGA from Texas Instruments,
a 10 Watt UV lamp emitting in the range 340 nm to 400 nm,
a controller to control mirrors of the DLP, such as a DDC 4100 Digital controller from Texas Instruments,
an interface and power supply for the above components, such as Texas Instruments DAD 2000 DMP Power and Reset Driver,
an optical system to focus light from the matrix projector at about 30 cm.

All the components are connected via a USB cable to a portable computer.

The computer is configured to:
enable the user to select an image from among an image or photograph bank,
access images via a network,
create or modify images.

The computer is also configured for controlling the matrix projector to project a selected image, either in visible light or invisible and UV light.

Before operating, the user sets the optical system to optically align the camera and the UV projector and to check the magnification so that the fields are superposed.

One may use photochrome products such as diarytlethene, for example those sold under references DAE-TZ, DAE-2BT. They may be used at concentrations ranging from about 0.001% to about 10% in weight, preferably with a total amount ranging form 0.1 to 3%.

The person to be treated applies the composition containing the photochrome(s) in an environment with no ambient UV light. Then, she projects an image on her skin after having selected an image, preferably in visible light. The image is focused and the person shifts to visible and UV light and stops the projection when she is satisfied with the color intensity result.

A UV protective composition can be applied to end the treatment.

### Example 10

In another example, a scanner is connected to the computer. The scanner is used to capture an image that is later projected onto the body.

The image that is projected may be selected from among various images:
that reproduce a make-up, for example a spot of a few centimeters to reproduce the effect of a blush, or a color surface to reproduce a lip make-up,
that reproduce natural effects, for example small imperfection to mimic freckles,
that reproduce decorations, such as images usually tattooed in a permanent or temporary fashion, and which may be nature illustrations such as animals or flowers, characters, letters, sentences, geometric patterns, etc.

The expression “comprising a” should be construed as being synonymous with “comprising at least one”.

The invention is not limited to the illustrated examples. In particular, zones of the body other than the face may be treated. All of the examples referring to treatment of the face are equally valid for the treatment of other regions.

1. A treatment method for treating human keratinous material, the method comprising: a) projecting an image onto a zone to be treated; b) modifying the content and/or adjusting the projected image; and c) cosmetically treating the zone as a function of the image as modified and/or adjusted in this way.

2. A method according to claim 1, the projected image defining the zone to be treated.

3. A method according to claim 1, the projected image including one or more reference points and/or outlines, and the treatment being performed as a function of the reference point(s) and/or the outline(s).

4. A method according to claim 1 including applying a thermally stable photochromic composition on the zone to be treated.

5. A method according to claim 4, wherein the light forming the projected image in step a) is selected so as not to activate the thermally stable photochromic composition, and wherein the method further comprises: d) irradiating the zone so as to excite the thermally stable photochromic composition as a function of the projected image in step a).

6. A method according to claim 5, the light being modified between steps a) and d), and a source of UV being used in step d).

7. A method according to claim 4 wherein the thermally stable photochromic composition is irradiated in such a manner as to create, on developing the thermally stable photochromic composition, at least one reference point and/or one outline.

8. A method according to claim 7, wherein the zone is treated using the reference point(s) and/or the outline(s) thus created.

9. A method according to claim 8, treatment comprising manually applying a makeup as a function of the reference point(s) and/or the outline(s).

10. A method according to claim 8, treatment being carried out by automatically detecting the reference point(s) and/or the outline(s).

11. A method according to claim 1, wherein any movement of the zone to be treated is detected and the projected image is modified automatically as a function of a movement of the zone to be treated during projection of the image.

12. A method according to claim 1, the projected image being produced by an electronic addressable matrix imager.

13. A method according to claim 1, the projected image being generated from at least one acquired image of the person to receive the treatment.

14. A method according to claim 1, the projected image taking account of distortions induced, on projection, by the relief of said zone.

15. A method according to claim 1, wherein an animated sequence of images is projected.
